SEQUENCE LISTING (22 sequences)

SEQ ID NO: 1 - protein, 548 aa, Arabidopsis thaliana
MGTLTSVAFAAAVNIRFRSFHRENIKTTITTLPKWQKRLCFSSTEDSHRFRIAKCLGNDENSNRDDSIGENGETHKSSVVKTATFEEEDEETSKSSSTTSSSNEFGSDKTSMPSTIDPTYSSFQIDSFKLMELLGPEKVDPADVKLIKDKLFGYSTFWVTKEEPFGDLGEGILFLGNLRGKKEDVFAKLQRKLVEVASDKYNLFMIEEPNSEGPDPRGGARVSFGLLRKEVSEPGPTTLWQYVIALILFLLTIGSSVELGIASQINRLPPEVVKYFTDPNAVEPPDMELLYPFVDAALPLAYGVLGILLFHELGHFLAAVPKKVKLSIPYFIPNITLGSFGAITQFKSILPDRSTKVDISLAGPFAGAALSVSMFAVGLFLSTEPDAANDLVQVPSMLFQGSLLLGLISRATLGYAALHAATVSIHPLVIAGWCGLTTTAFNMLPVGCLDGGRAVQGAFGKNALVTFGLSTYVMLGLRVLGGPLALPWGLYVLICQRTPEKPCLNDVTEVGTWRKALVGIALILVVLTLLPVWDELAEEVGIGLVTTF

SEQ ID NO: 2 - protein, 544 aa, Nicotiana tabacum
MGTLTSCSFSTMNIRFRLNPPVNHSFSRRIQLKRMSKRNFGRLIIRCSSGNGSSNNNGSSSDGKLEKDSSNLATVTEETTEERNGGGGASGVENDSEDSPVSISSRPTISTVGSTYNNFQVDSFKLMELLGPEKVDPSDVKIIKEKLFGYSTFWVTKEEPFGDLGEGILFLGNLRGKREDVFAKLQSQLSEIMGDKYNLFMVEEPNSEGPDPRGGPRVSFGMLRKEVSEPGPTTLWQYVIAFLLFLLTIGSSVELGIASQITRLPPEVVKYFTDPNAIEPPDMQLLLPFVDSAIPLAYGVLGVQLFHEIGHFLAAFPRNVKLSIPFFIPNITLGSFGAITQFKSILPDRKAKVDISLVGPFAGAALSSSMFAVGLLLSSNPAASGELVQVPSTLFQGSLLLGLISRATLGYGAMHGAMVSIHPLVIAGWCGLTTSAFNMLPVGCLDGGRAVQGAFGKGSLIGFGLATYTLLGLGVLGGPLSLPWGLYVLICQRTPEKPCLNDVTEVGTWRKAALGVAIFLVVLTLLPVWDELAEELGIGLVTSF

SEQ ID NO: 3 - protein, 547 aa, Nicotiana tabacum
MGTLTSCSFSSMNIRFRLNPPVNYTFSRRIQLKRMSKRNFGRLIIRCSSGSSGNGSSNDSGSSSDGKLEKDSSNLATVTEETTEERNGGGGASGVENDSDDSPVSISSRPTISTVGSTYNNFQVDSFKLMELLGPEKVDPSDVKFIKEKLFGYSTFWVTKEEPFGDLGEGILFLGNLRGKREDVFAKLQSQLSEIMGDKYNLFMVEEPNSEGPDPRGGPRVSFGMLRKEVSEPGPTTLWQYVIAFLLFLLTIGSSVELGIASQITRLPPEVVKYFTDPNAIEPPDMQLLLPFVDSALPLAYGVLGVQLFHEIGHFLAAFPRNVKLSIPFFIPNITLGSFGAITQFKSILPDRKAKVDISLAGPFAGAALSSSMFAVGLLLSSNPAAAGELVQVPSTLFQGSLLLGLISRATLGYGAMHGAMVSIHPLVIAGWCGLTTSAFNMLPVGCLDGGRAVQGAFGKGSLIGFGLATYTLLGLGVLGGPLSLPWGLYVLICQRTPEKPCLNDVTEVGNWRKAALGVAIFLVVLTLLPVWDELAEELGIGLVTSF

SEQ ID NO: 4 - DNA, 1644 bp, Nicotiana tabacum
atgggaacgctaacgagctgcagtttcagctcaatgaatataaggttccgtttgaatcctccagttaattacactttcagtcgaagaatccaattgaagagaatgtccaaacggaatttcggtcgattgattattaggtgtagtagcggtagtagtggcaatggcagtagcaatgacagtggtagcagtagcgatgggaaattggaaaaggattcttcaaatttggctacagttactgaagaaaccactgaagaaaggaacggcggcggtggcgccagcggtgtggaaaatgattcggatgattctccggtgtcaatttcttccagaccaacaatatccactgttggatcaacttataataatttccaagtagattcttttaagttgatggaacttcttggaccagaaaaggttgatcccagtgatgtgaagttcattaaggaaaagttatttggctactctactttttgggtgactaaagaagaaccatttggagatcttggagagggcattcttttccttgggaatcttagaggaaagagggaggatgtttttgccaaacttcagagtcagttatcagaaattatgggtgataagtacaacctgttcatggtggaggaacctaattcagaggggccagacccgcgtggtgggcccagagtcagctttggtatgctgcggaaagaagtttctgaaccaggtccaacaactctctggcaatatgtaattgcttttctgttgttccttctcactattggttcctctgtggagctaggaattgcatctcagataactcgccttcctcctgaggtagttaagtactttactgatccaaatgcaattgaaccaccagatatgcagcttttattaccgtttgtggattctgctttaccgttggcatatggtgtgctgggtgtgcagttatttcatgaaattgggcattttctggctgcatttccaaggaatgtgaaattaagcattcctttctttattccaaacatcactcttggaagctttggagcaatcactcagttcaaatctattcttcccgatcgcaaagcaaaggtagacatttctcttgcgggtccttttgctggtgctgcattgtcttcttccatgtttgcggttggcctgttactctcatccaatcctgctgctgctggagagttggttcaggttcctagcacacttttccagggctctttgcttctcgggcttattagcagagccactcttggttatggagcaatgcatggtgcaatggtttcaatccatcctcttgtgatagctggctggtgtggcttgactacatcggcttttaatatgctgccagttggatgtcttgatggtgggagagctgtgcagggagcctttgggaaaggatcacttattggttttggtttggcgacatacacacttctgggcttgggcgtgcttggtggacctctgtcacttccttggggattgtatgtgcttatatgtcagaggacaccggagaaaccatgcttgaatgatgtaacagaggtcggaaattggagaaaagcagctcttggtgtggctatattccttgttgtattgactcttcttcctgtatgggatgaacttgcagaagaactaggtataggtcttgtaaccagcttttga

SEQ ID NO: 5 - DNA, 1635 bp, Nicotiana tabacum
atgggaacgctaacgagctgcagtttcagcacaatgaatataaggttccgtttgaatcctccagttaatcacagtttcagtcgaagaatccaattgaagagaatgtccaaacggaatttcggtagattgattattaggtgtagtagtggaaatggcagtagcaataacaatggcagcagtagcgatgggaaattggaaaaggattcttcaaatttagctacagttactgaagaaaccactgaagaaaggaacggcggcggtggcgccagcggtgtggaaaatgattccgaggattctccggtgtcaatttcttccagaccaacaatatccacggttggatcaacttataataatttccaagtagattcttttaagttgatggaacttcttggaccagaaaaggttgatcccagtgatgtgaagataattaaggaaaagttatttggctactctactttttgggtgactaaagaagaaccatttggagatcttggagagggcattcttttccttgggaaccttagaggaaagagggaggatgtttttgccaaacttcagagtcagttatcagaaattatgggtgataagtacaacctgttcatggtggaagagcctaattcagaggggccagacccacgtggtgggcccagagttagctttggtatgctgcggaaagaagtttctgaaccaggtccaacaactctctggcaatatgtaattgcttttctgttgttccttctcacaattggttcctctgtggagctaggaattgcatctcagataactcgccttcctcctgaggtagttaagtactttacggatccaaatgcaattgaaccaccagatatgcagcttttactaccgtttgtggattctgctataccactggcatatggtgtgttgggcgtgcagttatttcatgaaattgggcattttctggctgcgtttccaaggaatgtgaaattaagcattcctttctttattccaaacatcactcttggaagctttggagcaatcactcagttcaaatctattcttcctgatcgaaaagcaaaggtagatatttcgcttgtgggtccttttgctggtgctgcattgtcttcttcaatgtttgcggttggcctgttactctcatccaatcctgctgcttctggagagttggttcaggttcctagcacacttttccagggatctttgcttcttgggcttattagcagagccactcttggttatggagcaatgcatggagcaatggtttcaatccatcctcttgtgattgctggctggtgtggtttgactacgtcggcttttaatatgctaccagttggatgtcttgatggtgggagagctgtgcagggagcctttgggaaaggatcacttattggttttggtttggcgacatacacacttctgggcttgggcgtgcttggtggacctctgtcacttccttggggattatatgtgcttatatgtcagaggacaccagagaaaccatgcttgaacgatgtaacagaggtcggaacttggagaaaagcagctcttggtgtggctatattccttgtagtattgactcttcttcctgtatgggatgaacttgcagaagaactaggtataggtcttgtaaccagcttttga

SEQ ID NO: 6 - protein, 4 aa, Artificial Sequence
  Motif
GNLR

SEQ ID NO: 7 - protein, 5 aa, Artificial Sequence
  Motif
  misc_feature (3)..(4): Xaa can be any naturally occurring amino acid
HEXXH

SEQ ID NO: 8 - protein, 10 aa, Artificial Sequence
  Motif
  misc_feature (2)..(3): Xaa can be any naturally occurring amino acid
  misc_feature (5)..(7): Xaa can be any naturally occurring amino acid
NXXPXXXLDG

SEQ ID NO: 9 - DNA, 15 bp, Artificial Sequence
  (P212) Yb2Yb2 sequence
attcagaggggccag

SEQ ID NO: 10 - protein, 42 aa, Artificial Sequence
  Nt_Yb1-TN90 sequence
KYNLFMVEEPNSEGPDPRGGPRVSFGMLRKEVSEPGPTTLWQ

SEQ ID NO: 11 - protein, 42 aa, Artificial Sequence; identical to SEQ ID NO: 10
  Nt_Yb1-K326 sequence

SEQ ID NO: 12 - protein, 42 aa, Artificial Sequence; identical to SEQ ID NO: 10
  Nt_Yb2-K326 sequence

SEQ ID NO: 13 - protein, 20 aa, Artificial Sequence
  Nt_Yb2-TN90 sequence
KYNLFMVEEPNSGPTWWAQS

SEQ ID NO: 14 - protein, 24 aa, Artificial Sequence
  Nt_Yb1-TN90 sequence
GPLVTSLGIVCAYMSEDTGETMLE

SEQ ID NO: 15 - protein, 41 aa, Artificial Sequence
  Nt_Yb1-K326 sequence
GPLSLPWGLYVLICQRTPEKPCLNDVTEVGNWRKAALGVAI

SEQ ID NO: 16 - protein, 41 aa, Artificial Sequence
  Nt_Yb2-K326 sequence
GPLSLPWGLYVLICQRTPEKPCLNDVTEVGTWRKAALGVAI

SEQ ID NO: 17 - protein, 559 aa, Gossypium raimondii
MGTLTSFSFNAVNLRFTSSSIFRQRVGVQVFGSRKLNTKLCYLIPKEVSREKFLKFRCFCATNNSNNDADYTQNDGEIKEDSSNTASSTESTVTTASPPEDKAVQEKRTSNELPPSLSSRPPNIAPLGSVYNDFQIDSFKLMELLGPEKVDPADVKLIKDKLFGYSTFWVTKEEPFGDLGEGILFLGNLRGKREDVFTKLQTQLAEIVGDKYNLFMVEEPDSEAPDPRGGPRVSFGLLRKEVSEPGPTTLWQYVIAILLFLLTIGSSVELGIASQINRLPPEVVKYFTDPNAVDPPDMELLYPFVESALPLAYGVLGVLLFHELGHFLAAFPKKVKLSIPFFIPNITLGSFGAITQFKSILPDRSTQVDISLAGPFAGAALSFSMFAVGLLLSSNPDAAGDLVQVPSMLFQGSLLLGLISRATLGYAAMHAATVSIHPLVIAGWCGLTTTAFNMLPVGCLDGGRAVQGAYGKGALVGFGLTTYTLLGLGVIGGPLSLPWGLYVLICQRTPEKPCLNDVTEVGTWRKTAVTVAVFLVVLTLLPVWDELAEELGIGLVSTF

SEQ ID NO: 18 - protein, 546 aa, Vitis vinifera
MGTLTSCSFSTATFRFRSRPVGNDFRQRIHLFNITSKNLCFLCSKKQVSSGSFGRFRCFSTNGNGDKNEGEESSLVKDSNSKTATMPEEAEEELDSDKDPPAPVSSRPPSISPIGPGYNNFQVDSFKLMELLGPEKVDPADVKLIKDKLFGYSTFWVTKEEPFGDLGEGILFLGNLRGKREEIFAKLQSQLTEIMGDKYNLFMVEEPNSDGLDPRGGPRVSFGMLRKEVSEPGPTTLWQYVIAFLLFLLTIGSSVELGIASQINRLPPDVVKYFTDPDAIEPPDMGLLFPFVESALPLAYGVLGVQLFHEVGHFLAAFPKKVKLSIPYFIPNITLGSFGAITQFKSILPDRRTKVDISLAGPFAGAALSCAMFSVGLLLSSNPDAAGDLVQVPSMLFQGSLLLGLISRATLGYAAMHAATVPIHPLVIAGWCGLTTSAFNMLPVGCLDGGRAVQGAFGKGALSGFGLTTYTLLGLGVLGGPLSLPWGLYVLICQRSPEKPCLNDVTEVGTWRKTVLTVAIFLVVLTLLPVWDELAEELGIGLVTTF

SEQ ID NO: 19 - protein, 547 aa, Solanum lycopersicum
MGTLTSCSFSIMNLRFRMNPPISCKFSRRIQMKRMSKRSLGRLIISCSSGSGGGGGGGSSSNDNGSSNDRKLEKDSSNLATVTEETAGERNSGGEASDSEDSSVSISSRPTISTVGSTYNNFQVDSFKLMELLGPEKVDPSEVKIIKEKLFGYSTFWVTKEEPFGDFGEGILFLGNLRGKREDVFAKLQSQLSEVMGDKYNLFMVEEPNSEGPDPRGGPRVSFGMLRKEVSEPGPTSLWQYVIAFLLFLLTIGSSVELGIASQITRLPPEVVKYFTDPNAIEPPDMQLLLPFVDSALPLAYGVLGVQLFHEIGHFLAAFPRNVKLSIPYFIPNITLGSFGAITQFKSILPDRKAKVDISLAGPFAGAALSSSMFAVGLLLSSNPSAAAELVQVPSTLFQGSLLLGLISRATLGYGAMHAAVVSIHPLVIAGWCGLTTSAFNMLPVGCLDGGRAVQGAFGKGSLVGFGLATYSLLGLGVLGGPLSLPWGLYVLICQRSPEKPCLNDVTEVGTWRKAALGVAIFLVLLTLLPVWDELAEELGIGLVTTF

SEQ ID NO: 20 - protein, 545 aa, Solanum tuberosum
MGTLTSCSFSTMNLRFRMNPPISCNFSRRIQMKRMSKRSFDRLIISCSSSGGGGGGSSSNDNGSSNDGKLEKDSSNLATVTEETAEERNGSGEASDSEDSSVSISSRPTISTVGSTYNNFQVDSFKLMELLGPEKVDPSDVKIIKEKLFGYSTFWVTKEEPFGDFGEGILFLGNLRGKSEDVFAKLQSQLSEVMGDKYNLFMVEEPNSEGPDPRGGPRVSFGMLRKEVSEPGPTSLWQYVIAFLLFLLTIGSSVELGIASQITRLPPEVVKYFTDPNAIEPPDMQLLVPFVDSALPLAYGVLGVQLFHEIGHFLAAFPRNVKLSIPYFIPNITLGSFGAITQFKSILPDRKAKVDISLAGPFAGAALSSSMFAVGLLLSSNPAAAAELIQVPSTLFQGSLLLGLISRATLGYGAMHGAMVSIHPLVIAGWCGLTTSAFNMLPVGCLDGGRAVQGAFGKGSLVGFGLATYSLLGLGVLGGPLSLPWGLYVLICQRSPEKPCLNDVTEVGTWRKAALGVAIFLVLLTLLPVWDELAEELGIGLVTTF

SEQ ID NO: 21 - DNA, 8911 bp, Nicotiana tabacum
acatttttcaccattgagaacaaaatcaactattgtttggtttttattcatcacaatgggaacgctaacgagctgcagtttcagctcaatgaatataaggttccgtttgaatcctccagttaattacactttcagtcgaagaatccaattgaagagaatgtccaaacggaatttcggtcgattgattattaggtgtagtagcggtagtagtggcaatggcagtagcaatgacagtggtagcagtagcgatgggaaattggaaaaggattcttcaaatttggctacagttactgaagaaaccactgaagaaaggaacggcggcggtggcgccagcggtgtggaaaatgattcggatgattctccggtgtcaatttcttccagagtcagtttctcttttgtttttgtttttaaaaaatctgagtttttttttccgtttttttagatattttaattttatgttagtgttgttattgtctaaatatttaagtttattgcttcaagttatatttttagaatttggtatggatgaaaggaggaaaatttggcgtaaacgaagagaagataagtgtatttaactgataactgatgatataagtaattaagtattagttattactagtttattgtgtgttgttctttatgcagctggttgttgtactatgatataggcttatgcagtaggagaacttgttcttccagctaggtgttcatatgcccttttccctggttgttgacttgcttatacggataggaaatacatttaaggaaatgaatggagagaaatattgaaaaatctgatcttgaatggcaaagcttaaaagcaatttgcctatttggcggaattattaaaagctatcacttcgtcatttaatgcaaatgaagcgatgcgaagcacaagagccatgcactctgaagaagtatgcgcttcaaacatgacacaaagtgatcctgacgagaatcacttgttctttgaatctcacatttgaggaattgcattagtatcaacattattgtaaataggatgctaaaaattagttacttaattgcgatttgattgttttagtactaaattcatatatgtttaatattttttaattctctgtaacttgcgtcacttcaaaggagcatgctcttcactactcgttttttgtttcaagtctcatggaccttattgcttctttgcatttttcactttagaaaacactaatattggaatcatttcaaaaacagcactgatactgggaaatcttacaactagtttcttttgaaattgcaataccgaaggcttttcacttttatataggagtactgtttatatttcaactaagtgactggtttaaaggaattgtggctggggtgtctatgaatttaagaaacttatctggtcattaagatagcaagaaaagtgtcaaataattatcctttattacattgtagtcagaaaatgttcaagtaatctctgttggctcataagaagctcgccacatgtattcctataatggtcaaaataaagagtagcttcatgatatgaagcaatgttttgaaaggcgttttcggggcgagccctggggcgaggcacaccaaaaatgcgtcggggcgatggtgtggggcgaaagtctcaagaggcatacgcccagcaattcggggcgtacgcccgggtgtttgaggcgagttgttttagtgaggcgtaagccccagagaggttttcaaattaaaacaaaatttgttgaataagcccttcatataatacccaaattttcaaaattcagcttggtaattactcaaaagttttaaaaggaactgaaatgtattaaatttaaaagtcaataccttttttattgattgaagccccaattcatggtcttttctaattctttatcttgtccgctagtctgctaatatctcccaaaagcaacgaatattcaatttttttttttacaaatacaaagagaactccattctctttcatagttcaaagttcaaattgaaggttagtcatcctttttgttcctccatgtagaaaactttattcttttcgactcaagttacttgagcttctaagtagcgtataatatattgttttgactagttttttgtggggatgaagcgcgcgcgcgcgcgcacaaacatatatatatatatatatatatatatatttcacatttttatagttttcttcattttttatgcaattttacctgtttataaatatttattgtaattatattattttataaaatattaaaaattaaatacctatagggcttacgcctcgcagaggcatatgtaaaacgtctcgccttacgccacgccttttaaaacattgatatgaagaaacattaaacagatgaagaggagaaaccaatgtactgaatgtgtttgctttcttggcagccaacaatatccactgttggatcaacttataataatttccaagtagattcttttaagttgatggaacttcttggaccagaaaaggttgatcccagtgatgtgaagttcattaaggaaaagttatttggctactctactttttgggtgactaaagaagaaccatttggagatcttggagagggcattcttttccttgggaatcttagaggaaagagggaggatgtttttgccaaacttcagagtcagttatcagaaattatgggtgataagtacaacctgttcatggtggaggaacctaattcagaggggccagacccgcgtggtgggcccagagtcagctttggtatgctgcggaaagaagtttctgaaccaggtccaacaactctctggcaatatgtaattgcttttctgttgttccttctcactattggttcctctgtggagctaggaattgcatctcaggtctgctcgttaaccacacagtttgactttaaagacattgtttcatttcttattcttctcataaaattctctattttttctaacaattgacttgatgacttaaatttatgttgagcagataactcgccttcctcctgaggtagttaagtactttactgatccaaatgcaattgaaccaccagatatgcagcttttattaccgtttgtggattctgctttaccgttggcatatggtgtgctgggtgtgcagttatttcatgtaagatttctttgttatccttttagttgatcgaatgaaattctctgtaatgaaggggtcagccaagtccaaaaggaacatgaaaatcttctctaaagttcccagttctgtcgctgattatgatagaatggtgtaatttaggttttggcttccttctttggcaatattgattacattagttaggttgactagggtttgattttcttcaaaaaagaataattagcttcgattttagtatttcttttactttgaagttttaagtttcaagttaaaataggatggtggacaaatattaaactagatagaaatactaaggagcacatttattggcctcagagtagaagctctgtgttaagttatagagcatttttactgctgaaaaggggaaaataatctcaacagaaaggttacttttaggggggaaaaaaacgaactgccaaaacaatgaagatgtcccggactgatttagctatgctagtttgctacaaaatgcaatgttaatcgtggaaaaaattgttagtctaataagtattgatagaatgcattaagacaactatcttacccctaagttcttttataggcacatttacaatgtatatatctgatgtgggacagtaccacatttaatgttaaacctgtatacctataaatctaaactagctatctaacacttaccctcaagcaggtgcatgaaagaagtagctatctaatacttcccctcaagcaggtgcataacatgtcgtatggaccaagcctgctacatatataattggttcatgaattggtgaggggcttggtgaataagttagcatgttaattgttcaacttcacaaacttagtgacgatttctaggaatatcttttctttggcaaatggtaatcaatctctatgtgtttgctcatctcatgatacataggattcgatttgatctgaagtgcagcttgaactttgaagtcaaaagtgactgtacattgctgccacatgcattcgaatacttggctgaaaaccatttctaggtattaggcaccttttaaattaatgtcaaacgtgatcagaagtagtagaaggaagcattagctataagttactacagacttatcacattgtagtcataagtactatatagtactgggctcagacatgtagaagggacaattgtatgtgtatattagataagcttagcttttgcatctctaaatgacatgtggcttaacattgctgtaattccgcgtgacagcttgaagtttacctttcattttcatttgcttttggaaaaaatttccctttctgccttgctcgtttgttgctgcagttctaccagagtagcttaattttaaaatatagtagaataaaacaagatgttaaactgaaaaagagatggtggttagtctctccttagatggttttataccttcatatgatcatttctgttcctccaaacttattcctccctgttcaattttacatgaagagttgatttgttgcaggaaattgggcattttctggctgcatttccaaggaatgtgaaattaagcattcctttctttattccaaacatcactcttggaagctttggagcaatcactcaggttagcataatttgttgtcacttgtcaatgcttgaaacagtaaagatggcagctgaggattctaaactttctgaaagatttgtgtaaatggatcaattattttgaattccccaatgtgctgtttgctttcaggagagccatgactgagtacattgtcattcacctggttttattaaaagatggaaaatgctggtaatatgcttttgaagtggttcttatgcatggatcttgttgtcattgttaaagcttaaaccctgggttataggaaacttaataggttcctttttgtgaagggggtagggtttacatttcatttctgcgcagaggcaggcctgttggcagaatgcatttactacagggaattggtgtaaagtgttcgagatatgctactagcaatttcttctggctgcagcctcctccaaacaatctagtataagtccttttgagaacacgaggatttccaagatggttctgtaatatacaatagtcattggacattaatcccttggggtgttaataataatattattcaattctcttttccttacccatttggttctcacattatctaggacttttgtcgttttagatttgttagttcaaatttgtgatagaggagtgaagtaagagttctaaatgtaggctgtgaattgaggagggggaaaagaagtagaagtggtggtggaggagaatcaaatgttatctttgcttattaagatgatgtgaatgcaactcttcaaatgatgttaatatgtaatatactcaatgcttgcccttgttaagtgtcacttattttgttccttatccaaaaaaaaaaagtgtcacttattttgttaaagcatatgtaacagatttgacaatggtagagtctagcatggagggaaaaatcattgtgaaggatctgtgctagattaatctacttcattaaaccttgtcattccgcctaattcatcatcactctattaagcataaaacatatgcattaatgatcttctcttggttcctgcagttcaaatctattcttcccgatcgcaaagcaaaggtagacatttctcttgcgggtccttttgctggtgctgcattgtcttcttccatgtttgcggttggcctgttactctcatccaatcctgctgctgctggagagttggttcaggttcctagcacacttttccagggctctttgcttctcgggcttattagcagagccactcttggttatgggtaagtttagccctccacagggagcgaaagcatgacgttcttggaaaataattttcaatatgtacattagagttatttttcttgttcgcttgaggagtaacctctttttgcttatttatttttggtgcacccacgagattcctatttgtatagaagattgggagtctttcatagaaaaccatgtctttttgtaaggttttctactttttggtatccttgtacacgggcaagtttttgcccctttttaatgaatttattaccttatcaaaaaaaaaaaaaaacgaaagaaatccctttgtcaaaacccctatatctgtaggctgatcactttgaagtgcataaatatagttgtttctaaaagaaaaccttaacagttatgtgcatgaaggccagggaactttttgctttttccaaattggcaatatattttcgtttcactatttcaaatctttagcatataacatttgagctaatgtgcattcattttcacagagcaatgcatggtgcaatggtttcaatccatcctcttgtgatagctggctggtaaatttgagtttgttgcaagtaattttgttggcttaagtttagcatgcttgctctaaaattcattttgtaggtgtggcttgactacatcggcttttaatatgctgccagttggatgtcttgatggtgggagagctgtgcaggtggcctttgcattctttattctttcaacttgttttcatgaacttgggacactgtcaagcttaataacaaagcgtaatcaattgcatcctatgaaatttgaatgatgacaagttgctaactttgacgtagactgttgttttcataatattagatgaataacggtgcaaagcttgaaaagatattactaatacctacgattctagtattgcaaacttgtgggctcctaacgagatagcggctgttgagacttcttagcatgcttagacaatttggtttcccgatatattttgctggctttttcgtgaagaattgtgacctcagaaaatgaagagcttcttggctaagtgataattggttgtcccagataaagtgtgacgactgtcaacttcattttttgtttctggcagggagcctttgggaaaggatcacttattggttttggtttggcgacatacacacttctgggcttgggcgtggtaagcagtcttttgtcatttgatattggtagaacttaaaagtaaatcttcacattgctcaaagttatgactaatatgttttaggccaaatacatagacaaccacctaaacttggcaccaatttccactttcgagcgtgaacaaccagaatttttgcttttagtgttttttcctctctttttcttctctctcctccaccgatctctcaaccttccccaatttctccataaacaaccatgaaccttttttgcccccttcaatcttactccaacgcacaggtgccatggtccctttcttctcaaccttatgtcactgtcttcctaaattttccactacaaaatacaaattcctctctttctgtttaactttctctatagatctttgaggacctcatataattacaaaaaagaaaaacttataggcgcctttttctcaataatttactgtttgtgcaatatgatggactttggaatagagagctacaccaaccccaccgccaccactgcaactgccgacggcaattccgattatgatattcctcagacgttgtcgtcttccggtgtttatcaaacccttcttctcattaaattcaatgactcactttgaaaattcaggcggctagggaaattggacacctcacataaatttctaattgttattgccgttatttcaccaacaccgtcaaactcctctgtgacatcctttgtttcagttccatcgttgcctcaccaacaccaccttcttcaactctccgccatcaaactccgaccgtaaaagcatttgccgtagccctgaaattttcagcacttgttttgtttcagagcaagataactctgaaatttgccggaaatagtgggggggggggggggggggctgttttatttgtttcatttgctcaaaatctttttttaactgaaaagacacttggcattttttattccttattctatcatgtaagagcgtgtgaatttcacgcaatgtattttgtgtacgggttagtcacaaagtgtttaataggtatagattgacttaagttaaagtgtctaataggaacaagtctgaatcgatgtgtttaagtggaaattggtgccaagtttaggtggttgtctatgtatttgccctgtgttttattacttcgatgtgcatatgtggatcaaatgtctatccttcatatatgcaatcgttgtccagtgtctatttgttccctcttctttgtcactccctttctgttaagttttcattagtattaggttggaaatctggaaccttttttacttatttcatgtgcgcattgaggtcaaatcacttacggtcttttacacatctgtcctttaccctttttgcttagtgttgcaaattgtacagtcacttaaaacattctgtttgtgctgctatcagcttggtggacctctgtcacttccttggggattgtatgtgcttatatgtcaggtaataaaaatacattgctttatgaagtattatgcttgtgtaataatatcgccgtcctatttcatggtatataagtgttagtcgtttacaagtaaaaatcttggatcgtgtgacgtacagaggacaccggagaaaccatgcttgaatgatgtaacagaggtcggaaattggagaaaagcagctcttggtgtggctatattccttgttgtattgactcttcttcctgtatgggatgaacttgcagaagaactaggtataggtcttgtaaccagcttttgattcattaaagatggagtcctgccaattttgatgtgggcatgggcttaaggagttggtatgcatcattttttaagtaatcatactgatcttttgac

SEQ ID NO: 22 - DNA, 7879 bp, Nicotiana tabacum
aatgaagatattctttcgttatatgctcatactcaaacctcaccatcttccaattgacatttttcaccattaagaacaaaatcaactattgtttggtttttgttcatcacaatgggaacgctaacgagctgcagtttcagcacaatgaatataaggttccgtttgaatcctccagttaatcacagtttcagtcgaagaatccaattgaagagaatgtccaaacggaatttcggtagattgattattaggtgtagtagtggaaatggcagtagcaataacaatggcagcagtagcgatgggaaattggaaaaggattcttcaaatttagctacagttactgaagaaaccactgaagaaaggaacggcggcggtggcgccagcggtgtggaaaatgattccgaggattctccggtgtcaatttcttccagagttagtttctcttttgtttttgttacaaaaatctgagcttttttattcgtttgtttattttttattttatgttagtgttgttattgtctaaattttcaagtttttttgcttctaattacatttttaaaatttgatgagaggtggaaaatttggggtaaaagaagagaagggaagtgtatttaactgataactgatgatacaagtaattaagtattactagtatattgtgtgtagttctttatgcagctggttgttgtactatgatataggcttatgcagtaggagaactgattcttccagctacgtgttcatatgcctcttttccctggttgttcccttgcttatacagataggaaataaagttaaggcaatgaatggagaggaatgttgaaaaatgtgatcttgaatagcaaaccttaaaagcaatttgcctattgggcagaatttttaaaagctatcacttcgttacttaaggcaaatgaagtgatgtgaagcgcaagagccatgcactttggagaagtgtgtgcttcaaacatgacacgcaaagtgatcctgacgagaagcacttgttctttgaatctcaaatttaaggaattgcattagtatcaaaattattgtaaatgggatgctaaaattagtcacttaattgcaatttgattatttttgtgccaaattcaaatatgtgatgcttttaattctctgtaacttgcgtcatttcgaaggagcaagctcttcaatactcgtttcttgtttcaagccccatggacctcatcacttctttgcgtttttcacttcagaaaacactaaaactggaaccatttcaaaaaaagcactgatactgggaaatgttacaactagtttcttttgaaattgcaataggcttttcacttctatatactgtttttcaactaagtgaccggtttaaaagaattgtggctggggtatctatgaattttaagaaacttatctggtcattaagatagcaagaaaagtgtcaaatagttaactttattacattgtagtcagaaaatgttcaagtaatctctgttgacttataagaagctcgtcacatgcattcctgtaatggtcaaaataaagagtagctgcatgatgtgaagaaacattaaagagatgaagatgagaaaccaatgtactgaatgtgtttaatttcttggcagccaacaatatccacggttggatcaacttataataatttccaagtagattcttttaagttgatggaacttcttggaccagaaaaggttgatcccagtgatgtgaagataattaaggaaaagttatttggctactctactttttgggtgactaaagaagaaccatttggagatcttggagagggcattcttttccttgggaatcttagaggaaagagggaggatgtttttgccaaacttcagagtcagttatcagaaattatgggtgataagtacaacctgttcatggtggaagagcctaattcagaggggccagacccacgtggtgggcccagagttagctttggtatgctgcggaaagaagtttctgaaccaggtccaacaactctctggcaatatgtaattgcttttctgttgttccttctcacaattggttcctctgtggagctaggaattgcatctcaggtgaactcgtcaaccacgcagtttcactttaaaacattgtttcatttcttattcttctcataaaattctctatttctttaacaattaagttgacgactaaaatttatgttgaacagataactcgccttcctcctgaggtagttaagtactttacggatccaaatgcaattgaaccaccagatatgcagcttttactaccgtttgtggattctgctataccactggcatatggtgtgttgggcgtgcagttatttcatgtaagatttttttgttatccttttagttgattgaatggaattctctgtaatggaggagtcagccaagaccaaaagaaacatgaaaatcttctcaaaagttcccagatctgtctctgactatgatagaatggtgttatttaggcttttgatttccttctttggcaatattgattacattagttaggttgactagggtttgattttcttcattagctttgattttagtgtttcttttactttgaagttttaagtttcaggttaaaattggatggtgggcaaatattaaactagatagaaatactaaaggaacacatttattggcctcagagtagaagcttaatgttaagttataaagcatgtttgctactgaaaaggagaaaaataatctcaacagaaaggttaccgttagggaaaaagacgaagtgacaaaacaatgaagatgcactttactgatttagctatgctagtttgccacaaaatgcaatgttaatcatgaaaaattgttagtctaataagaattgagagaatgcgttaagacaactgtcttacccctacgttcttttataggcacacattacaaagcatacatctgatgtgggacgctatcacatttagtgttacacctgtattcctttacagcttgtttggatagttgttatgtattgtttcataatgtgtcgtatcgtattgtattgtatccttttgatgcatacaatgtttggataaattgtattgtttgtcgtcgtttcatgatgtcatgtaccaacaatatgaagaataaacttgcaatttacaaagaaaaagtaaggtacgaagtagaattattatataggaaggtagggtaaaggataaaatagtattatttaataataagaaacggcaagataagaggaaaaaacaaggtaacgacgcgaccaccgaaataggtcgttacataaagcattttttgtcgttacgtaacgagggatttaatgatacaatacaataaaatttaagtaacaatcaaaacaaatattgtatttaaagtaacaatacgatgcaatacaataggtaacacttaccctcaaacgggtgcatgaaagaagtagctagctaatacttcccatcaagcaggtgcataacatgttgtatggaccaagcctgctacatatacaattggttcatgaattgttgaggagcttggtgaataagttagcaagttagttgctcatctgcacaaacttagtgatgatttttaagaacatcttttctttggcaaatggtgatcaatctctacgtgtttgctcatctcatgaaacataggattcgattgatatgaagtgcagcttgaattttgaaaacagcttcagctggcctagggcattatcttggttgagtcggagatgtgggacctaaaagttgaagtaaccgtgtcgaggtattgaaccaacaactaagattacagaatcagacagaacttgtctgtggttaggaaatatatgaaagatcaaggctattgactaatctaatccaactggataattgatttacattgctgccacatacatttgaatacttggcatgaaaaccatttcttagtgttaggcaccttctaaattaaagtcaaaagtgatcagaagtaacagaaggaagcattagctataacttactacagacttatcaaatagtcaaaagtattctataatgctgggctcagacatgtagaagggacatttctatgtatatattagctaagcttagcttttgcatctctagatgacatgtggcttaacattgttgtagctccgtgtgacgactagaagttgctgctatgacattttcatttgcttttggagaaagtttacctttctgccttgcttgtttgtcgctgcagttctaccaaagtagcataattttaaaatacagaataaaacaagatgttaaactgaaaatgagatggcggttagtctctcctgaaatggttttataccttcatttctgttcttcagaacttattcctccctgttcaatttttacatgaagagttgatttgttgcaggaaattgggcattttctggctgcgtttccaaggaatgtgaaattaagcattcctttctttattccaaacatcactcttggaagctttggagcaatcactcaggttagcatatttttctgtcacttgtcaatgcgtgaaacagtaaagatggcagctgaggattctgaactttctgaaatgtttgtgcaaatggatcaattattttgaattcctcaaagtgctgtttgcttttaggagagccgtgactgagtacattgtcattcaccaggttttactaaaagatggaacatgctggtgatatccatgaatcttgttttcattgttaaagcttaaaccctggattataggaaacttaatgggttcctctttgtgaagggggtaggggttacatttcatttctgcccagaggcaggcctgatggcagaatgcatttactaaagggaattggtgtagagtgttgagctatgctactagcaatttcttctggctgcagcctcctccaaacaatctactataaatcctctaagaacacgaggagttccaagatggttctgtagtatacaatagtcaatggacattaatcccttggagcgttaataataagtattattcaatttccttttccttacccatttggtggtcacattatctaggacttttgtcaatttagcttcttttttttttagttcaagcttgtgatagaggactggaataaaagttctaaatgtaggctgtgattggggagaagagaaaagaagtagaattggtggtggaggagaatcaaatgctatctttgctgattgagatgctgtgaatgtaacgcctaaatcaatgttaatctgtaatacactggcatgctttcctttcttaagtatcacatgtcttgttaaggctgtgattgaggagaagagaaaagaagtagaattggtggtggaggagaatcaaatgttatctttgctgattgagatgctgtgaatgtaacacctaaatcaatgttaatgcataatacactggcatgctttcccttcttaagtatcagatgttttcttaaagtcatgcgtagcatatttgacaatggtgtagtctagcatggaggggaaattttttgtgaaggatctgtgctagattaatctacttcatcaaaccttgtcattccgcctaatcatcgtcactccatcaatcataaaacatatgcatcaatgttcttctcttgcttcctgcagttcaaatctattcttcctgatcgaaaagcaaaggtagatatttcgcttgtgggtccttttgctggtgctgcattgtcttcttcaatgtttgcggttggcctgttactctcatccaatcctgctgcttctggagagttggttcaggttcctagcacacttttccagggatctttgcttcttgggcttattagcagagccactcttggttatgggtaagtttagtcctccacagggagcaaaagcatgaagttcttggaaaacaattttcagaatgtacatttgagttatttttctactataatatgaagattcccttcgtcaaaacccctatatccgtaggccgatcactttgaagtgcatatatatagttgtttttaaaagaaacccttaacgattatgtccatgaaggccaggattttttttctttttccacaatggcaatatatttttgtttcacttattcgaatcgttagcatataatatttgagctaatgtgcattattttcacagagcaatgcatggagcaatggtttcaatccatcctcttgtgattgctggctggtaaatttgagtttgttgtaagtaattttgttggcttaagtttaggatgcttgcttaaatttcattttgcaggtgtggtttgactacgtcggcttttaatatgctaccagttggatgtcttgatggtgggagagctgtgcaggtggcctttgcattcttaatttatcaatttgttttcatgaacttgggacactgtcaaggttaatatcatagcataatcgacagcatcctactaaattcaaatgaatgacaagtcagtaaccttgaagtggactgttgtttacatagtattagatgattgaatggtgcaaagcttgaaaagataatactaatagacgtacaagtcctatctgattctcgtattggaaacatgtgggtctcctagcaagataatagctgttgagactttttagcatgcttgagaagtttggtttcctttgcattcttaatttatcaatttgttttcatgaacttaggacactgtcaaggttaataacaaagcataatcaattgcatcctattaaatttggatgatgacaagttgctcactttgaaatagactgttgtttacatagtgttagatgattaacggtacaaagcttgaaaggatattactaatagatgtgtggatcctacgtgattctagtattgcaaacttgtggggcaacgaaataggggctgttgagactttttagtatgcttagagaaattggtttcccgaaatatactcttttatgaagaattgtgatctcagaaaataaagagctttcttggctaagagataactggttgtcccagctaaagtatgatgactgtcaacttcattttttgtttctggcagggagcctttgggaaaggatcacttattggttttggtttggcgacatacacacttctgggcttgggcgtggtaagcagtcttttgtcatttgatatagggtagaacttaaaagtaaatcttcacattgctcaaagtttcgactaatatgttttgttactttgtagatcaaatgtctgtccttcatatatccaatttgttcccccttctgttaagttttaattagtattaggttggcagtctggaccctttttttacttatttcatgtgcccattgaggtcaaatcacttatggtcttttacacatctgtcctttaccctttttgcttggtgttgcaaattgtacaatgagtcacttaaaacattctgtttgtgctgctatcagcttggtggacctctgtcacttccttggggattatatgtgcttatatgtcaggtaacaaaaatatgttgctttatgaaatattatgcttgtgtaataatatcgccatcctatttcatggtatataagtgttagtcgtttacaagttaaaatcttggatcatgtgacgtacagaggacaccagagaaaccatgcttgaacgatgtaacagaggtcggaacttggagaaaagcagctcttggtgtggctatattccttgtagtattgactcttcttcctgtatgggatgaacttgcagaagaactaggtataggtcttgtaaccagcttttgattcattaaagatggagtcctgccaattttgatgtgggcatgggtttaaggagttggtatgcatcattttaagtattcatactgatcttttgactgtacgtagtggactatgtatacatgtatatagtgaggtaccattttttcactgcagatttattttaataaacgtggaaatttct

(19) United States
(12) Patent Application Publication
(10) Pub. No.: US 2019/0203218 A1
(43) Pub. Date: Jul. 4, 2019

(54) METHOD

(71) Applicant: North Carolina State University, Raleigh, NC (US)

(72) Inventors: Katherine Drake Stowe, Raleigh, NC (US); Ramsey S. Lewis, Raleigh, NC (US); Matthew Edward Humphry, London (GB); Kieron Edwards, London (GB)

(21) Appl. No.: 16/312,057

(22) PCT Filed: Jun. 21, 2017
(86) PCT No.: PCT/US2017/038419

§ 371 Date: Dec. 20, 2018

Related U.S. Application Data
(60) Provisional application No. 62/353,130, filed on Jun. 22, 2016.

(51) Int. Cl. C12N 15/82; C07K 14/415; C12Q 1/6895; A24B 13/00; A24B 15/24; A24B 3/12; A24B 15/18; A24B 7/00

(52) U.S. Cl. CPC C12N 15/8243; C07K 14/415; C12Q 1/6895; A24B 13/00; C12Q 2600/13; A24B 3/12; A24B 15/183; A24B 7/00; A24B 15/243; A24B 15/245

(57) ABSTRACT

The present invention provides a method for increasing nitrogen-use efficiency and/or nitrogen-utilisation efficiency in a plant comprising modifying the plant by increasing the activity or expression of an ethylene-dependent gravitropism-deficient and yellow green protein (EGY) in said plant.

### FIELD OF THE INVENTION

The present invention relates to methods for the improvement of nitrogen-use efficiency and/or nitrogen-utilisation efficiency in plants and, in particular, to methods for reducing tobacco-specific nitrosamines or their precursors in tobacco plants. The present invention further relates to plants and their downstream products (e.g. propagation materials, harvested leaf, processed leaf and consumable products) obtainable by the present methods.

### BACKGROUND

The growing world population demands increasing food production. According to the projections of the United Nations Food and Agricultural Organization (FAO, 2006, “World Agriculture: towards 2030/2050”. Interim Report. FAO, Rome, Italy), the production of cereals will need to increase by 60% from 2000 to 2050. More crop production requires greater amounts of plant nutrients. As a consequence, fertilizer consumption will increase considerably as the requirement for crop production increases. Fertilizers are applied to balance the gap between the permanent export of nutrients from the field with the harvested crops and the nutrients supplied by the soil. However, not all of the applied fertilizer ends up in the crop. Part of the fertilizer nutrients are lost to the wider environment and can also contribute to environmental problems.

As such, there are societal and political requirements for more efficient use of plant nutrients in agriculture.

Nitrogen (N) and nitrate (NO₃⁻) availability regulate many aspects of plant metabolism, growth, and development. Nitrogen use efficiency (NUE) and nitrogen utilization efficiency (NUTL) are key indicators for assessing the efficacy of measures to decrease nitrogen (N) losses while maintaining agricultural productivity. NUE is typically defined as the weight of harvested plant material per unit of applied nitrogen. NUTL is typically defined as the weight of harvested material per unit of nitrogen that is accumulated by the plant.

Improvements in NUE and/or NUTL are therefore a desirable aim for the agricultural industry.

Tobacco-specific nitrosamines (TSNA) are formed from nicotine and related compounds by nitrosation reactions that occur during the curing and processing of tobacco, as illustrated in FIG. 1. The nitrosating agent in air-cured tobacco is usually nitrite derived from the reduction of leaf nitrate by the action of microbes during curing, and production of nitrosamines has been shown to correlate to high levels of nitrate/nitrite and nitric oxide (Liang S., Yang J., Zhou J., Yu J., Ma Y., Bai R., Xu F., Yang C; Acta Physiol. Plant. (2013) 35: 3027-3036). Application of exogenous substances can reduce tobacco-specific nitrosamine levels by regulating biosynthesis of alkaloids and nitrite in burley tobacco. Acta Physiol. Plant. (2013) 35: 3027-3036). TSNA reduction is a desirable aim for the tobacco industry.

### SUMMARY OF THE INVENTION

According to a first aspect, the present invention provides a method for increasing nitrogen-use efficiency (NUE) and/or nitrogen utilization efficiency (NUTL) in a plant comprising modifying the plant by increasing the activity or expression of an ethylene-dependent gravitropism-deficient and yellow green protein (EGY) in said plant.

In another aspect, the present invention provides the use of increased expression of a polynucleotide encoding an EGY protein or increased activity of an EGY protein in a plant for increasing nitrogen-use efficiency and/or nitrogen-utilisation efficiency in a plant.

In another aspect the present invention provides a method for reducing a level of at least one tobacco-specific nitrosamine (TSNA) or a precursor thereto in a tobacco plant by increasing the activity or expression of an ethylene-dependent gravitropism-deficient and yellow green protein (EGY) in said plant.

In another aspect the present invention provides a method for producing a tobacco plant, a tobacco plant propagation material, a tobacco leaf, a cut harvested tobacco leaf, a processed tobacco leaf or a cut and processed tobacco leaf which has a reduction in at least one TSNA or a precursor thereto, the method comprising modifying said tobacco plant to increase expression of an EGY gene or increase activity of an EGY protein in said plant.

In a further aspect the present invention provides a construct or vector comprising a nucleic acid encoding an EGY protein.

In another aspect the present invention provides a plant cell (e.g. a tobacco plant cell):
i) comprising an exogenous EGY polynucleotide;
ii) comprising a construct or vector according to present invention; and/or
iii) obtainable (e.g obtained) by a method or use of the present invention.

In another aspect the present invention provides a plant (e.g. a tobacco plant):
i) comprising an exogenous EGY polynucleotide;
ii) which has been modified to achieve an increase in nitrogen-use efficiency and/or nitrogen-utilisation efficiency in comparison to an unmodified plant, wherein the modification is an increase in activity or expression of an EGY polypeptide in said modified plant;
iii) obtainable by a method or use of the present invention;
iv) comprising a construct or vector of the present invention; and/or
v) comprising a cell of the present invention.

In a further aspect the present invention provides a plant propagation material (e.g. a plant seed) obtainable from a plant of the present invention.

In a further aspect the present invention provides a harvested leaf of a plant (e.g. a tobacco plant) according to the present invention or obtainable from a propagation material according to the present invention or obtainable from a method according to the present invention.

In another aspect the present invention provides a processed leaf (e.g. a processed tobacco leaf, preferably a non-viable processed tobacco leaf):
a. comprising a plant cell according the present invention;
b. obtainable from a plant obtainable from a method according to the present invention
c. obtainable from processing a plant according to the present invention;
d. obtainable from a plant propagated from a plant propagation material according to the present invention; and/or
e. obtainable by processing a harvested leaf according to the present invention.

In a further aspect the present invention provides a plant product:
a. prepared from a plant obtained or obtainable by the method according to the present invention;
b. prepared from a plant according to the present invention;
c. prepared from a plant propagated from the plant propagation material according to the present invention;
d. prepared from a harvested leaf according the present invention;
e. prepared from a processed leaf according to the present invention; and/or
f. prepared from or comprising a plant extract obtained or obtainable from a modified plant according to the present invention.

In another aspect the present invention provides a smoking article, smokeless tobacco product or tobacco heating device comprising a plant or a portion thereof from the species Nicotiana tabacum or Nicotiana rustica obtained or obtainable by the according to method according to the present invention or a tobacco plant according to the present invention.

In a further aspect the present invention provides use of a
a. a plant according to the present invention or a part thereof;
b. a plant (preferably the leaves) propagated from a plant propagation material according to the present invention;
c. a harvested leaf according to the present invention; and/or
d. a processed leaf according to the present invention;
e. a plant extract obtained from a plant according to the present invention, for production of a product of the present invention.

The present invention further provides use of a plant according to the present invention for breeding a plant.

The present invention further provides use of a plant according to the present invention to grow a crop.

The present invention further provides use of a plant according to the present invention to produce a leaf (e.g. a processed (preferably cured) leaf).

In a further aspect the present invention provides a method for screening a plant (e.g. a tobacco plant) for an EGY variant which comprises comparing an EGY polynucleotide sequence (e.g. gene) in said plant to a wild-type EGY polynucleotide sequence to identify the presence of a variant EGY polynucleotide sequence in said plant, and optionally further determining that said variant EGY polynucleotide sequence is associated with increased NUE and/or increased NUTL.

In another aspect the present invention provides a method for identifying a plant (e.g. a tobacco plant) with a predisposition to increased NUE and/or increased NUTL which comprises comparing an EGY polynucleotide sequence (e.g. gene) in said plant to a wild-type EGY polynucleotide sequence to identify the presence of a variant EGY polynucleotide sequence in said plant, and optionally further determining that said variant EGY polynucleotide sequence is associated with increased NUE and/or increased NUTL.

The present invention further provides a method, leaf, plant, plant propagation material, a harvested leaf, a product, a use or a combination thereof substantially as described herein with reference to the description and drawings.

### BRIEF DESCRIPTION OF THE DRAWINGS

FIG. 1 shows the formation of tobacco-specific nitrosamines (TSNA) from precursors such as nicotine and nornicotine via a nitrosation reaction in tobacco smoke.

FIG. 2 shows the positional cloning of the (A) Yb1 gene and (B) Yb2 gene. Dark lines are drawn to represent relative positions of genetic markers. The genetic distance between these markers is shown in cM. Grey rectangles indicate exons. (C) Consequences of Yb mutations. The 8 bp deletion in exon 2 of Yb2 results in a frame shift that leads to the incorporation of 8 spurious amino acids before terminating in a premature stop codon. The insertion of a T in exon 9 of Yb1 results in a frame shift that leads to the incorporation of 21 spurious amino acids before terminating in a premature stop codon.

FIG. 3 shows polypeptide and polynucleotide sequences for Arabidopsis EGY1 (NP_198372) and Nicotiana tabacum EGY1 (NtEGY1) and EGY2 (NtEGY2).

FIG. 4 shows amino acid alignments of Yb related proteins. Alignments were generated using MUSCLE. Conserved nucleotides are indicated by shaded squares. The three conserved domains, GNLR, HEXXH, and NPDG are underlined with black lines. Accession numbers are as follows: Arabidopsis EGY1 (NP_198372), Gossypium raimondii predicted EGY1 (XP_012492480), Vitis vinifera predicted EGY1 (XP_002269447), S. lycopersicum L2 (NP_001299816), S. tuberosum predicted EGY1 (XP_006339202).

FIG. 5 shows mean values for yield, nitrogen use efficiency (N-USE), nitrogen utilization efficiency (N-UTL), and nitrate levels for non-transformed burley tobacco cultivar TN 90LC, and transgenic (green stem) and non-transgenic (white stem) R₁ progeny of Ro TN 90LC plants transformed with 35S:NtYb1. Means for R₁ progeny are averaged over three transgenic events (20 measured individuals per genotypic class per transgenic event).

FIG. 6 shows the effect of NtYb₁ overexpression in burley tobacco cultivar TN 90LC on TSNA accumulation in air cured leaves. Total TSNAs are calculated as the sum of NNN, NAT, NAB, and NNK. ‘n’=equals the number of plants sampled.

### DETAILED DESCRIPTION OF THE INVENTION

### Nitrogen-Use Efficiency (NUE or N-USE)/Nitrogen-Utilisation Efficiency (NUTL or N-UTL)

Nitrogen is one of the major nutritional elements required for plant growth and is commonly the rate limiting element in plant growth. Nitrogen is part of numerous important compounds found in living cells, such as amino acids, proteins (e.g. enzymes), nucleic acids, and chlorophyll. 1.5% to 2% of plant dry matter is nitrogen and approximately 16% of total plant protein. Thus, the availability of nitrogen has a major impact on amino acid synthesis as well as amino acid composition, accumulation of amino acids, on protein synthesis and accumulation thereof, and based thereupon it is a major limiting factor for plant growth and yield (Frink et al.; Proc. Natl. Acad Sci. USA96, 1175 (1999), which is incorporated herein by reference).

Plants can utilize a wide range of nitrogen species including volatile ammonia (NH₃), nitrogen oxides (NO_{X}), mineral nitrogen, like nitrate (NO₃⁻) and ammonium salts (NH₄⁺), urea and urea derivates, and organic nitrogen (amino acids, peptides, and the like). Some plants are able to utilize atmospheric nitrogen by symbiotic bacteria or certain fungi. However, in most agricultural soils, nitrate (NO₃⁻) is the most important source of nitrogen (Crawford N. M., Glass A. D. M.,

Trends in Plant Science, 3 389 (1998); Hirsch R. E., Sussman M. R., TIBTech 17, 356 (1999), incorporated herein by reference). Nevertheless also ammonium NH₄₊ plays an important, probably underestimated role, because most plants preferentially take up NH₄⁺ when both forms are present-even if NH₄⁺ is present at lower concentrations than NO₃⁻ (von Wiren N. et al; Curr. Opin. Plant Bioi. 3, 254 (2000), incorporated herein by reference).

In one embodiment, carrying out a method and or use of the invention results in an increase in NUE and/or NUTL in the modified plant when compared to a plant which has not been modified to increase the activity or expression of an EGY protein.

In one embodiment, carrying out a method and or use of the invention results in an increase in NUE in the modified plant when compared to a plant which has not been modified to increase the activity or expression of an EGY protein.

In one embodiment, carrying out a method and or use of the invention results in an increase in NUTL in the modified plant when compared to a plant which has not been modified to increase the activity or expression of an EGY protein.

NUE is typically defined as the weight of harvested plant material per unit of applied nitrogen. NUTL is typically defined as the weight of harvested material per unit of nitrogen that is accumulated by the plant.

Various suitable methods for calculating NUE are known in the art (e.g. Carranca et al.; Farming for Food and Water Security; 2012; pp. 57-82; Weih et al.; Plant Soil 2011, 339, 513-520; Weih; Agronomy 2014, 4(4), 470-477—which are incorporated herein by reference).

Accordingly, NUE and NUTL may be calculated using any suitable method known in the art.

Suitably NUE may be calculated according to the following method (as described in Lewis et al.; J. Agric. Food Chem. 60:6454-6461(2012); Moll et al.; Agron. J.; 74, 562-564; (1982)—each of which are incorporated herein by reference):

N-use efficiency (N-USE, kg kg⁻¹)=(cured leaf yield, kg ha⁻¹/units N fertilizer, kg ha⁻¹),

Suitably NUE may also be calculated according to the following method:

N-use efficiency (N-USE, g g⁻¹)=(plant biomass, gram plant⁻¹/units N fertilizer, gram plant⁻¹)

Suitably NUTL may be calculated according to the following method (as described in Lewis et al.; J. Agric. Food Chem. 60:6454-6461(2012); Moll et al.; Agron. J.; 74, 562-564; (1982)—each of which are incorporated herein by reference):

N-utilization efficiency (N-UTL, kg kg⁻¹)=(cured leaf yield, kg ha⁻¹/N-ACC, kg ha⁻¹),

where N-ACC=(cured leaf yield, kg ha⁻¹×total N concentration, g kg⁻¹)

Suitably NUTL may also be calculated according to the following method:

N-utilization efficiency (N-UTL, g g⁻¹)=(plant biomass, g plant⁻¹/N-ACC, g plant⁻¹)

where N-ACC=(plant biomass, g plant⁻¹×% N*10)/1000

Suitably, total nitrogen may be calculated according to the method described in Nelson and Sommers (Agron. J. 65:109-112;1973—incorporated herein by reference) and nitrate accumulation (NO₃—N) may be calculated according to CORESTA Recommended Method No. 36 (2011)—which is incorporated herein by reference.

The above method for calculating NUTL is based on total nitrogen in the leaves only, and not total nitrogen for all plant parts. Suitably, in one embodiment tissue nitrogen concentrations for stalk, midribs, and roots are proportional to leaf nitrogen concentration. In one embodiment the NUE may be increased by at least about 1%, at least about 2%, at least about 3%, at least about 4%, at least about 5%, at least about 6%, at least about 7%, at least about 8%, at least about 9%, at least about 10%, at least about 15% or at least about 20%.

In some embodiments the NUE may be increased by between about 1% and about 20%, by between about 1% and about 15%, by between 1% and about 10%, by between 2% and about 10%, or by between about 2% and 5%.

In one embodiment the NUTL may be increased by at least about 1%, at least about 2%, at least about 3%, at least about 4%, at least about 5%, at least about 6%, at least about 7%, at least about 8%, at least about 9%, at least about 10%, at least about 15% or at least about 20%.

In some embodiments the NUTL may be increased by between about 1% and about 20%, by between about 1% and about 15%, by between 1% and about 10%, by between 2% and about 10%, or by between about 2% and 5%.

In one embodiment, the term “increasing NUE” and/or “increasing NUTL” means that the modified plant exhibits a generally enhanced yield under normal conditions or under nitrogen deficient conditions.

The term “yield” as used herein generally refers to a measurable produce from a plant, particularly a crop. Yield and yield increase (in comparison to a non-modified plant) can be measured in a number of ways, and it is understood that a skilled person will be able to apply the correct meaning in view of the particular embodiments, the particular crop concerned and the specific purpose or application concerned. In the preferred embodiments of the present invention described herein, an increase in yield refers to increased biomass yield, increased seed yield, and/or increased yield regarding one or more specific content(s) of a whole plant or parts thereof or plant seed(s). Suitably, “yield” refers to biomass yield comprising dry weight biomass yield and/or fresh weight biomass yield, each with regard to the aerial and/or underground parts of a plant, depending on the specific circumstances (test conditions, specific crop of interest, application of interest, and the like). In each case, biomass yield may be calculated as fresh weight, dry weight or a moisture adjusted basis, and on the other hand on a per plant basis or in relation to a specific area (e.g. biomass yield per acre/square meter/or the like). Suitably, “yield” refers to seed yield which can be measured by one or more of the following parameters: number of seed or number of filled seed (per plant or per area (acre/square meter or the like)); seed filling rate (ratio between number of filled seeds and total number of seeds); number of flowers per plant; seed biomass or total seed weight (per plant or per area (acre/square meter or the like); thousand kernel weight (TKW; extrapolated from the number of filled seeds counted and their total weight; an increase in TKW may be caused by an increased seed size, an increased seed weight; or other parameters allowing to measure seed yield. Seed yield may be determined on a dry weight or on a fresh weight basis, or typically on a moisture adjusted basis, e.g. at 15.5 percent moisture.

In a preferred embodiment, yield refers to the biomass of a harvestable product.

Suitably, the biomass may be dry biomass.

Suitably, the biomass may be aerial biomass, preferably dry aerial biomass.

Suitably, the biomass may be fresh weight biomass, for example aerial fresh weight biomass.

Suitably, the biomass may refer to the harvestable parts of the plant.

The term “aerial” as used herein means the part(s) of the plant above the ground and may include the leaves, the fruit, the seed and the stem. In one embodiment the term aerial parts of the plant as used herein means leaf only or leaf and/or stem of the plant. Preferably, the term “aerial” is used to refer to the leaves of the plant.

In one embodiment the term “enhanced biomass” means that the plant exhibits an increased growth rate under conditions of limited nitrogen supply, compared to the corresponding wild-type photosynthetic active organism. An increased growth rate may be reflected inter alia by an increased biomass production of the whole plant, or by an increased biomass production of the aerial parts of a plant, or by an increased biomass production of the underground parts of a plant, or by an increased biomass production of parts of a plant, like stems, leaves, blossoms, fruits, seeds.

In an embodiment thereof, increased biomass production includes higher fruit yields, higher seed yields, higher fresh matter production, and/or higher dry matter production.

In one embodiment the biomass may be increased by at least about 1%, at least about 2%, at least about 3%, at least about 4%, at least about 5%, at least about 6%, at least about 7%, at least about 8%, at least about 9%, at least about 10%, at least about 15% or at least about 20%. In some embodiments the biomass may be increased by between about 1% and about 20%, by between about 1% and about 15%, by between 1% and about 10%, by between 2% and about 10%, or by between about 2% and 5%.

### Ethylene-Dependent Gravitropism-Deficient and Yellow Green Protein (EGY)

Ethylene-dependent gravitropism-deficient and yellow green protein (EGY) is a membrane-associated and ATP-independent metalloprotease required for development of both thylakoid grana and well-organized lamellae in chloroplast. It is required for the accumulation of chlorophyll and chlorophyll a/b binding (CAB) proteins in chloroplast membranes, and for grana formation and normal chloroplast development. It is involved in the regulation of nuclear gene expression in response to ammonium stress and interacts with ABA signalling and carries out beta-casein degradation in an ATP-independent manner in vitro.

The terms “EGY protein” and “EGY polypeptide” are used herein to refer to a protein which comprises conserved GNLR, HEXXH and NXXPXXXLDG motifs. In one embodiment, the terms “EGY protein” and “EGY polypeptide” are used to refer to a protein which comprises GNLR, HEXXH and NXXPXXXLDG motifs and provides an ATP-independent metalloprotease activity.

The term “increasing the activity or expression of an EGY protein” means that the expression or activity of an EGY protein in the plant as a whole is increased in the modified plant when compared to a plant that has not been modified to increase the activity or expression of an EGY protein.

In one embodiment the increase in activity or expression of the EGY protein in the modified plant may be more than about 5% when compared to an unmodified plant. Suitably the increase in EGY protein expression or activity may be more than about 10%, suitably more than about 15%. In some embodiments the increase in EGY protein activity or expression may be more than about 20%, more suitably more than about 30%. In some embodiments increase in EGY protein activity or expression may be more than about 40%, such as about 50%, about 60%, about 70%, about 80%, about 90% or about 100%.

In one embodiment, the present methods and/or uses may comprise increasing the expression or activity of more than one EGY polynucleotide, gene or protein.

The one or more EGY polynucleotide, gene or protein may be any EGY polynucleotide, gene or protein as described herein.

Suitably, the present methods and/or uses may comprise increasing the expression of NtEGY1 and/or ntEGY2.

In one embodiment, an EGY variant may be an EGY polypeptide which has an increased activity compared to a wild-type EGY polypeptide.

Suitably, a wild-type EGY polypeptide may be e.g. an EGY polypeptide shown as Nicotiana EGY1 or EGY2 sequence, e.g. comprising one of the sequences shown as SEQ ID NO:2 or 3, Arabidopsis EGY1 (AtEGY) (NP_198372), Gossypium raimondii EGY1 (GrEGY1) (XP_012492480), Vitis vinifera EGY1 (VvEGY1) (XP_002269447), S. lycopersicum L2 (SI L2) (NP_001299816) or S. tuberosum EGY1 (St EGY1) (XP_006339202).

Suitably, a wild-type EGY polypeptide may comprise a sequence shown as SEQ ID NO: 1, 2, 3, 17, 18, 19 or 20. In one embodiment, a wild-type EGY polypeptide may comprise a sequence shown as SEQ ID NO: 2 or 3.

A variant EGY polypeptide may have a level of activity which is increased by at least 5%, at least 10%, at least 15%, at least 20%, at least 25%, at least 30%, at least 40% or at least 50% compared to a wild-type EGY polypeptide.

A variant EGY polypeptide with increased activity compared to a wild-type EGY polypeptide may increase NUE and/or NUTL by a greater amount than a wild-type EGY protein when expressed in a plant.

Suitably, a variant EGY polypeptide may increase NUE and/or NUTL by at least 5%, at least 10%, at least 15%, at least 20%, at least 25%, at least 30%, at least 40% or at least 50% more than a wild-type EGY polypeptide when expressed in a plant.

In some embodiments the modification of the plant to increase activity or expression of the EGY protein may be carried out using gene-editing.

Gene-editing may be carried out using any method known in the art. A few non-limiting examples are presented here including use of the CRISPR-Cas9 system. CRISPR/Cas9 genomic editing tools are available commercially such as “Guide-it” from Clontech (Avenue du President Kennedy 78100 Saint-Germain-en-Laye, France). Another method of gene-editing includes the use of TALEN (transcription activator-like effector nuclease) technology with kits available commercially (e.g. from Addgene, 1 Kendall Sq. Ste. B7102, Cambridge, Mass. 02139, USA). A further method comprises the use of Zinc Finger Nucleases such as the CompoZr® Zinc Finger Nuclease Technology available from Sigma-Aldrich. Another method comprises the use of meganucleases (or a further method) described in Silva et al Curr Gene Ther. February 2011; 11(1): 11-27, WO2007/047859 and WO2009/059195 (the teachings of which are incorporated herein by reference). A yet further method is oligonucleotide-directed mutagenesis (ODM) such as KeyBase® available from Keygene (Agro Business Park 90, 6708 PW Wageningen, The Netherlands).

In one embodiment the EGY protein and/or a polynucleotide encoding the EGY protein (e.g. an EGY gene) for use in accordance with the present invention may be endogenous to the plant (e.g. the tobacco plant).

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

In another embodiment the EGY protein for use in accordance with the present invention may be exogenous to the plant (e.g. the tobacco plant).

Examples of EGY proteins are Nicotiana EGY1 or EGY2 (e.g. comprising SEQ ID NO: 2 and 3 respectively), Arabidopsis EGY1 (AtEGY) (NP_198372), Gossypium raimondii EGY1 (GrEGY1) (XP_012492480), Vitis vinifera EGY1 (VvEGY1) (XP_002269447), S. lycopersicum L2 (SI L2) (NP_001299816), S. tuberosum EGY1 (St EGY1) (XP_006339202).

In one embodiment, the EGY polypeptide comprises the following amino acid sequences:
i) GNLR (SEQ ID NO: 6)
ii) HEXXH wherein X is any amino acid (SEQ ID NO: 7)
iii) NXXPXXXLDG wherein X is any amino acid (SEQ ID NO: 8)

In one embodiment, the EGY polypeptide comprises the following conserved residues: a GNLR motif comprising Gly-176, Asn-177, Leu-178 and Arg-179; a HEXXH motif comprising His-311, Glu-312, X-313, X-314 and His-311; and a NXXPXXXLDG motif comprising Asn-441, X-442, X-443, Pro-444, X-445, X-446, X-447, Leu-448, Asp-449 and Gly-450; wherein X is any amino acid and wherein the numbering of the conserved residues means the effective equivalent position with the EGY protein is aligned with the Arabidopsis thaliana EGY1 protein having SEQ ID NO: 1.

In one embodiment, the EGY polypeptide comprises a polypeptide shown as SEQ ID NO: 2 or SEQ ID NO: 3 or a functional fragment thereof or a sequence which has at least 70% sequence identity to SEQ ID NO: 2 or 3 or a functional fragment thereof.

Suitably the polypeptide may have at least 80% identity with SEQ ID NO: 2 or SEQ ID NO: 3 or a functional fragment thereof.

More suitable the polypeptide may have at least 90% identity with SEQ ID NO: 2 or SEQ ID NO: 3 or a functional fragment thereof. Preferably the polypeptide may have at least 95% identity with SEQ ID NO: 2 or SEQ ID NO: 3 or a functional fragment thereof.

More preferably the polypeptide may have at least 99% identity with SEQ ID NO: 2 or SEQ ID NO: 3 or a functional fragment thereof.

The term “functional fragment” as used herein refers to a portion of a polypeptide that is capable of encoding an EGY protein that retains its activity. In one embodiment the functional fragment may be a portion of a polypeptide of the invention comprising at least 50 amino acids, at least 75 amino acids, at least 100 amino acids, at least 150 amino acids, at least 200 amino acids, at least 300 amino acids, at least 400 amino acids or at least 500 amino acids of an EGY polypeptide as described herein.

In one embodiment, the EGY polypeptide comprises a polypeptide shown as SEQ ID NO: 2 or SEQ ID NO: 3 or a sequence which has at least 70% sequence identity to SEQ ID NO: 2 or 3. Suitably the polypeptide may have at least 80% identity with SEQ ID NO: 2 or SEQ ID NO: 3. More suitable the polypeptide may have at least 90% identity with SEQ ID NO: 2 or SEQ ID NO: 3. Preferably the polypeptide may have at least 95% identity with SEQ ID NO: 2 or SEQ ID NO: 3. More preferably the polypeptide may have at least 99% identity with SEQ ID NO: 2 or SEQ ID NO: 3.

In one embodiment the EGY polynucleotide may be NtEGY1, NtEGY2, AtEGY (NM_122913.3), GrEGY1 (XM_012637026), VvEGY1 (XM_002269411.2), SI L2 (NM_001312887.1) or StEGY1 (XM_006339140.2).

Suitably the EGY gene or polynucleotide is NtEGY1 and/or NtEGY2.

In one embodiment NtEGY1 comprises SEQ ID NO: 4 or a sequence which has at least 70% sequence identity to SEQ ID NO: 4. Suitably NtEGY1 may have at least 80% identity with SEQ ID NO: 4. More suitable NtEGY1 may have at least 90% identity with SEQ ID NO: 4. Preferably NtEGY1 may have at least 95% identity with SEQ ID NO: 4. More preferably NtEGY1 may have at least 99% identity with SEQ ID NO: 4.

In one embodiment NtEGY1 comprises or consists of SEQ ID NO: 4.

In one embodiment an NtEGY1 gene is a genomic polynucleotide sequence which is transcribed into a polynucleotide sequence defined herein as NtEGY1. In one embodiment an NtEGY1 genomic polynucleotide sequence comprises SEQ ID NO: 21 or a sequence which has at least 70% sequence identity to SEQ ID NO: 21. Suitably NtEGY1 may have at least 80% identity with SEQ ID NO: 21. More suitable NtEGY1 may have at least 90% identity with SEQ ID NO: 21. Preferably NtEGY1 may have at least 95% identity with SEQ ID NO: 21. More preferably NtEGY1 may have at least 99% identity with SEQ ID NO: 21.

In one embodiment NtEGY1 comprises or consists of SEQ ID NO: 21.

In one embodiment NtEGY2 comprises SEQ ID NO: 5 or a sequence which has at least 70% sequence identity to SEQ ID NO: 5. Suitably NtEGY2 may have at least 80% identity with SEQ ID NO: 5. More suitable NtEGY2 may have at least 90% identity with SEQ ID NO: 5. Preferably NtEGY2 may have at least 95% identity with SEQ ID NO: 5. More preferably NtEGY2 may have at least 99% identity with SEQ ID NO: 5.

In one embodiment NtEGY2 comprises or consists of SEQ ID NO: 5.

In one embodiment an NtEGY2 gene is a genomic polynucleotide sequence which is transcribed into a polynucleotide sequence defined herein as NtEGY2. In one embodiment an NtEGY2 polynucleotide sequence comprises SEQ ID NO: 22 or a sequence which has at least 70% sequence identity to SEQ ID NO: 22. Suitably NtEGY2 may have at least 80% identity with SEQ ID NO: 22. More suitable NtEGY2 may have at least 90% identity with SEQ ID NO: 22.

Preferably NtEGY2 may have at least 95% identity with SEQ ID NO: 22. More preferably NtEGY2 may have at least 99% identity with SEQ ID NO: 22.

In one embodiment NtEGY2 comprises or consists of SEQ ID NO: 22.

In one embodiment the EGY gene or polynucleotide comprises SEQ ID NO: 4, 5, 21 or 22 or a functional fragment thereof; or a variant of SEQ ID NO: 4, 5, 21 or 22 or a functional fragment thereof which has at least 70% sequence identity to SEQ ID NO: 4, 5, 21 or 22 or a functional fragment thereof. Suitably a variant may have at least 80% sequence identity to SEQ ID NO: 4, 5, 21 or 22 or a functional fragment thereof. Suitably a variant may have at least 90% sequence identity to SEQ ID NO: 4, 5, 21 or 22 or a functional fragment thereof. Suitably a variant may have at least 95% sequence identity to SEQ ID NO: 4, 5, 21 or 22 or a functional. Suitably a variant may have at least 99% sequence identity to SEQ ID NO: 4, 5, 21 or 22 or a functional fragment thereof.

In a further embodiment the EGY polypeptide for use in accordance with the present invention may be encoded by a polynucleotide sequence comprising:
i) a polynucleotide sequence shown herein as SEQ ID NO: 4, SEQ ID NO: 5, SEQ ID NO: 21 or SEQ ID NO: 22;
ii) a functional fragment of the polynucleotide sequence shown in i) which functional fragment encodes an EGY polypeptide, or
iii) a polynucleotide which encodes a polypeptide comprising the amino acid sequence shown herein as SEQ ID NO: 2 or SEQ ID NO: 3; or
iv) a polynucleotide sequence which can hybridize to the polynucleotide taught in i), ii) or iii) above under high stringency conditions, or
v) a polynucleotide sequence which has at least 70% (preferably 85%, more preferably 90%) identity with the polynucleotide shown in i), ii) or iii) above, or
vi) a polynucleotide sequence which differs from a polynucleotide shown in i), ii) or iii) due to degeneracy of the genetic code.

The term “degeneracy of the genetic code” as used herein refers to the redundancy in codons encoding polypeptide sequences exhibited as the multiplicity of three-codon combinations specifying an amino acid. For example in an mRNA molecule encoding a polypeptide having an isoleucine amino acid, isoleucine can be encoded by AUU, AUC or AUA. This means that a DNA molecule encoding the RNA can have multiple sequences yet the resulting polypeptide will have the same sequence. In other words polymorphic nucleotide sequences can encode the same polypeptide product. This means that one nucleic acid sequence can comprise a sequence with very low sequence identity to a second sequence while encoding the same polypeptide sequence.

In one embodiment the polynucleotide sequence may have at least 80% identity with SEQ ID NO: 4, 5, 21 or 22. Suitably the polynucleotide sequence may have at least 90% identity with SEQ ID NO: 4, 5, 21 or 22. Suitably the polynucleotide sequence may have at least 95% identity (more suitably at least 99% identity) with SEQ ID NO: 4, 5, 21 or 22. More suitably the polynucleotide sequence may have at least 99% identity with SEQ ID NO: 4, 5, 21 or 22.

In one embodiment the polynucleotide sequence may have at least 80% identity with SEQ ID NO: 4 or SEQ ID NO: 5. Suitably the polynucleotide sequence may have at least 90% identity with SEQ ID NO: 4 or SEQ ID NO: 5. Suitably the polynucleotide sequence may have at least 95% identity (more suitably at least 99% identity) with SEQ ID NO: 4 or SEQ ID NO: 5. More suitably the polynucleotide sequence may have at least 99% identity with SEQ ID NO: 4 or SEQ ID NO: 5.

In one embodiment, an EGY variant may be an EGY polypeptide which has a reduced activity compared to a wild-type EGY polypeptide.

Suitably, a wild-type EGY polypeptide may be e.g. an EGY polypeptide shown as Nicotiana EGY1 or EGY2 sequence, e.g. comprising one of the sequences shown as SEQ ID NO:2 or 3, Arabidopsis EGY1 (AtEGY) (NP_198372), Gossypium raimondii EGY1 (GrEGY1) (XP_012492480), Vitis vinifera EGY1 (VvEGY1) (XP_002269447), S. lycopersicum L2 (SI L2) (NP_001299816) or S. tuberosum EGY1 (St EGY1) (XP_006339202).

Suitably, a wild-type EGY polypeptide may comprise a sequence shown as SEQ ID NO: 1, 2, 3, 17, 18, 19 or 20. In one embodiment, a wild-type EGY polypeptide may comprise a sequence shown as SEQ ID NO: 2 or 3.

An EGY variant with reduced activity may be generated using a variety of methods which are known in the art. By of example, a polynucleotide sequence encoding a wild-type EGY polypeptide may be modified to reduce the activity of the EGY protein by gene-editing. Gene-editing may be carried out using any method known in the art. A few non-limiting examples are presented here including use of the CRISPR-Cas9 system. CRISPR/Cas9 genomic editing tools are available commercially such as “Guide-it” from Clontech (Avenue du President Kennedy 78100 Saint-Germain-en-Laye, France). Another method of gene-editing includes the use of TALEN (transcription activator-like effector nuclease) technology with kits available commercially (e.g. from Addgene, 1 Kendall Sq. Ste. B7102, Cambridge, Mass. 02139, USA). A further method comprises the use of Zinc Finger Nucleases such as the CompoZr® Zinc Finger Nuclease Technology available from Sigma-Aldrich. Another method comprises the use of meganucleases (or a further method) described in Silva et al Curr Gene Ther. February 2011; 11(1): 11-27, WO2007/047859 and WO2009/059195 (the teachings of which are incorporated herein by reference). A yet further method is oligonucleotide-directed mutagenesis (ODM) such as KeyBase® available from Keygene (Agro Business Park 90, 6708 PW Wageningen, The Netherlands).

A variant EGY polypeptide may have a level of activity which is reduced by at least 5%, at least 10%, at least 15%, at least 20%, at least 25%, at least 30%, at least 40% or at least 50% compared to a wild-type EGY polypeptide.

A variant EGY polypeptide with reduced activity compared to a wild-type EGY polypeptide may increase NUE and/or NUTL by a lower amount than a wild-type EGY protein when expressed in a plant.

Suitably, a variant EGY polypeptide may increase NUE and/or NUTL by at least 5%, at least 10%, at least 15%, at least 20%, at least 25%, at least 30%, at least 40% or at least 50% less than a wild-type EGY polypeptide when expressed in a plant.

Suitably a EGY variant with reduced activity may be used to increase NUE and/or NUTL in a tobacco variety such as for example Yellow Burley in order to increase NUE and/or NUTL whilst retaining the sensory characteristics of the tobacco variety (e.g. the Yellow Burley variety).

NUE and/or NUTL may be determined by any suitable method as described herein.

### Tobacco-Specific Nitrosamine (TSNA)

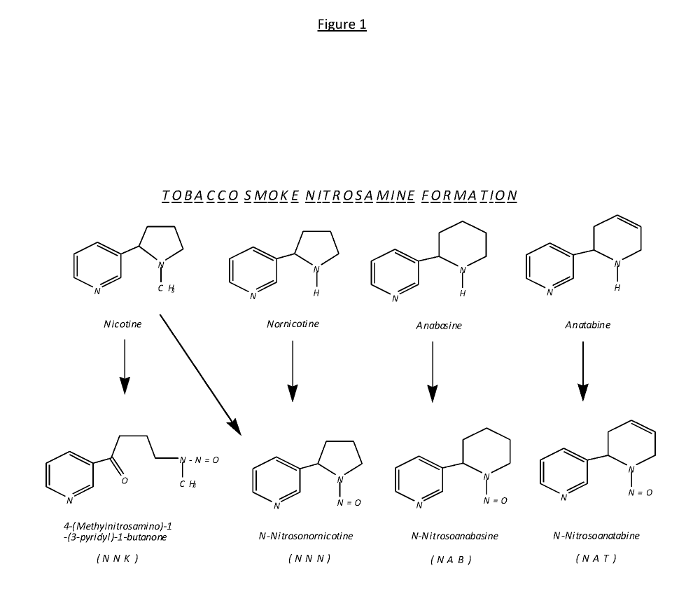

It is well known that residual nitrogen in tobacco leaves contributes to the formation of nitrosamines, through a nitrosation reaction as illustrated in FIG. 1. In particular, nitrate and nitrite and nitric oxide (NO) act as precursors to tobacco-specific nitrosamine (TSNA) formation in cured leaf. Without wishing to be bound by theory, the present inventors consider that the modification of nitrate assimilation by modulation of EGY protein expression provides the capacity to modulate the production of nitrite and nitric oxide (NO) and therefore the levels of TSNA which form during tobacco leaf curing and processing. In particular, by increasing the expression and/or activity an EGY protein expression the pool of free NO₃—N available for reduction to nitrite (the primary nitrosating agent for TSNA formation in air-cured tobacco) by plant nitrate reductase enzymes or via microbial activity is reduced and TSNA levels are therefore reduced.

Accordingly, in one aspect the present invention provides a method for reducing a level of at least one tobacco-specific nitrosamine (TSNA) or a precursor thereto in a tobacco plant (e.g. a tobacco plant leaf) comprising modifying the plant by increasing the activity or expression of an ethylene-dependent gravitropism-deficient and yellow green protein (EGY) in said plant.

The present invention also provides the use of increased expression of an EGY gene or increased activity of an EGY protein for the reduction of at least one TSNA or a precursor thereto in a tobacco plant.

A reduction of TSNA content or concentration in a tobacco product prepared from a tobacco plant which has increased expression of an EGY gene or increased activity of an EGY protein is a highly advantageous technical effect.

In one embodiment there is provided a method for producing a tobacco plant, a tobacco plant propagation material, a tobacco leaf, a processed tobacco leaf, a cut tobacco leaf or a cut and processed tobacco leaf which has a reduction in at least one TSNA or a precursor thereto, the method comprising modifying said tobacco plant to increase expression of an EGY gene or increase activity of an EGY protein.

The term “tobacco-specific nitrosamine” or “TSNA” as used herein has its usual meaning in the art, namely a nitrosamine which is found only in tobacco products or other nicotine-containing products. Suitably the at least one tobacco-specific nitrosamine may be 4-(methylnitrosamino)-1-(3-pyridyl)-1-butanone (NNK), N′-nitrosonornicotine (NNN), N′-nitrosoanatabine (NAT) or N-nitrosoanabasine (NAB).

More suitably the at least one tobacco-specific nitrosamine may be NNK or NNN.

The term “precursor thereto” when used in relation to at least one tobacco-specific nitrosamine refers to one or more chemicals or compounds of a tobacco plant that give rise to the formation of a tobacco-specific nitrosamine or are involved in the nitrosation reaction leading to tobacco-specific nitrosamine production. Suitably the term “precursor thereto” may refer to nitrate, nitrite or nitric oxide. Suitably the term “precursor thereto” may refer to nitrate.

In one embodiment carrying out a method and or use of the invention results in a reduction of at least one TSNA or a precursor thereto in the modified tobacco plant when compared to a tobacco plant which has not been modified to increase the activity or expression of an EGY protein.

The terms “reducing at least one TSNA or precursor thereto” or “reduction of at least one TSNA or precursor thereto” are used herein to mean that the concentration and/or total content of the at least one TSNA or precursor thereto in the product, method or use of the invention is lower in relation to a comparable product, method or use. For example, a comparable tobacco product would be derived from a tobacco plant which had not been modified according to the present invention, but in which all other relevant features were the same (e.g. plant species, growing conditions, method of processing tobacco, etc).

Any method known in the art for determining the concentration and/or levels of at least one TSNA or precursor thereto may be used. In particular a method such as that detailed in Example 4 herein may be used. For example when determining the concentration and/or level of a precursor to a tobacco-specific nitrosamine a method such as one detailed in WO2009/022183, in Morot-Gaudry-Talarmain et al. 2002. Planta, 215:708-715 or in Mur et al Plant Science 181 (2011) 509-519 (which are incorporated herein by reference) may be used.

Suitably the concentration and/or total content of the at least one tobacco-specific nitrosamine or precursor thereto may be reduced by carrying out a method and/or use of the present invention. Suitably the concentration and/or level of the at least one tobacco-specific nitrosamine or precursor thereto may be reduced in a tobacco plant of the invention (e.g. obtainable or obtained by a method and/or use of the invention) when compared to the concentration and/or level of the at least one tobacco-specific nitrosamine(s) or precursor thereto in a tobacco plant which has not been modified to increase activity or expression of an EGY protein.

The concentration and/or total content of the at least one tobacco-specific nitrosamine(s) or precursor thereto may be reduced in a tobacco leaf, harvested leaf, processed tobacco leaf, tobacco product or combinations thereof obtainable or obtained from a tobacco plant of the invention when compared with a tobacco leaf, harvested leaf, processed tobacco leaf, tobacco product or combinations thereof obtainable or obtained from a tobacco plant which has not been modified to increase activity or expression of an EGY protein.

Suitably the concentration and/or total content of the at least one tobacco-specific nitrosamine or precursor thereto may be reduced in a tobacco plant leaf. Suitably the concentration and/or total content of the at least one tobacco-specific nitrosamine or precursor thereto may be reduced in a processed tobacco leaf.

Suitably the concentration and/or level of the at least one tobacco-specific nitrosamine or precursor thereto may be reduced in a tobacco product.

In one embodiment the at least one tobacco-specific nitrosamine or precursor thereto may be reduced by at least about 1%, at least about 3%, at least about 5%, at least about 10%, at least about 20%, at least about 30%, at least about 40%, at least about 50%, at least about 60%, at least about 70%, at least about 80% or at least about 90%. In some embodiments the at least one tobacco-specific nitrosamine or precursor thereto may be reduced by between about 5% and about 95%, by between about 10% and about 90%, by between 20% and about 80%, by between 30% and about 70%, or by between about 40% and 60%.

In relation to processed (e.g. cured) tobacco leaf, the at least one tobacco-specific nitrosamine or precursor thereto may be reduced by between about 5000 ng/g and about 50 ng/g, by between about 4000 ng/g and about 100 ng/g, by between about 3000 ng/g and 500 ng/g or by between 2000 ng/g and 1000 ng/g. In some embodiments the at least one tobacco-specific nitrosamine or precursor thereto may be reduced by at least about 5000 ng/g, at least about 4000 ng/g, at least about 3000 ng/g, at least about 2000 ng/g, at least about 1000 ng/g, at least about 500 ng/g, at least about 100 ng/g or at least about 50 ng/g.

### Increasing Expression

The method and uses of the present invention comprise an increase in expression of at least one EGY protein. The increase in expression can be achieved by any means known to the person skilled in the art.

The term “increased expression” or “overexpression” as used herein means any form of expression that is additional to the original wild-type expression level.

“Increased expression” means that a plant is increased in the mRNA level or the protein level in comparison with an expression level of a parent plant of the same breed. The expression level is compared to a corresponding part in the parent plant of the same breed cultured under the same condition. A case where the expression level increases at least 1.1 times greater than that of the parent plant is preferably considered as a case where the expression level is increased. Here, it is more preferable that the expression level of the plant has a significant difference of 5% by a t-test compared with that of the parent plant, in order to be considered that there is an increase in the expression level. It is preferable that the expression levels of the plant and the parent plant be measured at the same time by the same method. However, data stored as background data may be also used.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, U.S. Pat. No. 5,565,350; WO9322443, which are incorporated herein by reference), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3¹ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1: 1183-1200, which is incorporated herein by reference). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994), which is incorporated herein by reference.

In one embodiment the increased expression may be achieved by the use of gene-editing or targeted mutagenesis.

Gene-editing may be carried out using any method known in the art. A few non-limiting examples are presented here including use of the CRISPR-Cas9 system. CRISPR/Cas9 genomic editing tools are available commercially such as “Guide-it” from Clontech (Avenue du President Kennedy 78100 Saint-Germain-en-Laye, France). Another method of gene-editing includes the use of TALEN (transcription activator-like effector nuclease) technology with kits available commercially (e.g. from Addgene, 1 Kendall Sq. Ste. B7102, Cambridge, Mass. 02139, USA). A further method comprises the use of Zinc Finger Nucleases such as the CompoZr® Zinc Finger Nuclease Technology available from Sigma-Aldrich. Another method comprises the use of meganucleases (or a further method) described in Silva et al Curr Gene Ther. February 2011; 11(1): 11-27, WO2007/047859 and WO2009/059195 (the teachings of which are incorporated herein by reference). A yet further method is oligonucleotide-directed mutagenesis (ODM) such as KeyBase® available from Keygene (Agro Business Park 90, 6708 PW Wageningen, The Netherlands).

Suitably, gene-editing may be used to alter the sequence of an EGY gene promoter in vivo.

In another embodiment of the invention, increased expression of an EGY polypeptide may be achieved by expressing within the plant a polynucleotide (e.g. an exogenous polynucleotide) comprising a nucleic acid sequence encoding an EGY polypeptide. In one embodiment, said polynucleotide (e.g. exogenous polynucleotide) comprises a nucleic acid sequence encoding a an EGY polypeptide operably linked with a heterologous promoter for directing transcription of said nucleic acid sequence in said plant.

In some embodiments the promoter may be selected from the group consisting of: a constitutive promoter, a senescence-specific promoter, a tissue-specific promoter, a developmentally-regulated promoter and an inducible promoter.

In one embodiment the promoter may be a constitutive promoter.

A constitutive promoter directs the expression of a gene throughout the various parts of a plant continuously during plant development, although the gene may not be expressed at the same level in all cell types. Examples of known constitutive promoters include those associated with the cauliflower mosaic virus 35S transcript (Odell J T, Nagy F, Chua N H. (1985). Identification of DNA sequences required for activity of the cauliflower mosaic virus 35S promoter. Nature. 313 810-2, which is incorporated herein by reference), the rice actin 1 gene (Zhang W, McElroy D, Wu R. (1991). Analysis of rice Act1 5′ region activity in transgenic rice plants. Plant Cell 3 1155-65, which is incorporated herein by reference) and the maize ubiquitin 1 gene (Cornejo M J, Luth D, Blankenship K M, Anderson O D, Blechl A E. (1993). Activity of a maize ubiquitin promoter in transgenic rice. Plant Molec. Biol. 23 567-81, which is incorporated herein by reference). Constitutive promoters such as the Carnation Etched Ring Virus (CERV) promoter (Hull R, Sadler J, LongstaffM (1986) The sequence of carnation etched ring virus DNA: comparison with cauliflower mosaic virus and retroviruses. EMBO Journal, 5(2):3083-3090, which is incorporated herein by reference).

The constitutive promoter may be selected from a: a carnation etched ring virus (CERV) promoter, a cauliflower mosaic virus (CaMV 35S promoter), a promoter from the rice actin 1 gene or the maize ubiquitin 1 gene.

Suitably the promoter may be a CERV promoter.

In one embodiment the promoter may be a senescence-specific promoter.

A “senescence-specific promoter” (SAG) can be a promoter which is associated with controlling the expression of a senescence-associated gene. Hence, the promoter can restrict expression of a coding sequence (i.e. a gene) to which it is operably linked substantially exclusively in senescing tissue. Therefore, a senescence-specific promoter can be a promoter capable of preferentially promoting gene expression in a plant tissue in a developmentally-regulated manner such that expression of a 3′ protein-coding region occurs substantially only when the plant tissue is undergoing senescence. It will be appreciated that senescence tends to occur in the older parts of the plant, such as the older leaves, and not in the younger parts of the plants, such as the seeds.

One example of a plant which is known to express numerous senescence-associated genes is Arabidopsis. Hence, a senescence-specific promoter may be isolated from a senescence-associated gene in Arabidopsis. Gepstein et al. (The Plant Journal, 2003, 36, 629-642, which is incorporated herein by reference) conducted a detailed study of SAGs and their promoters using Arabidopsis as a model. The genetic construct may comprise a promoter from any of the SAGs disclosed in this paper. For example, a suitable promoter may be selected from a group consisting of SAG12, SAG13, SAG101, SAG21 and SAG18, or a functional variant or a functional fragment thereof.

In one embodiment the promoter may be a SAG12 promoter, which will be known to the skilled technician, or a functional variant or a fragment thereof (Gan & Amasino, 1997, Plant Physiology, 113: 313-319, which is incorporated herein by reference.

Suitable promoters and sequences thereof may be found in WO2010/097623 which is incorporated herein by reference.

In another embodiment the promoter may be a tissue-specific promoter.

A tissue-specific promoter is one which directs the expression of a gene in one (or a few) parts of a plant, usually throughout the lifetime of those plant parts. The category of tissue-specific promoter commonly also includes promoters whose specificity is not absolute, i.e. they may also direct expression at a lower level in tissues other than the preferred tissue.

A number of tissue-specific promoters are known in the art and include those associated with the patatin gene expressed in potato tuber and the high molecular weight glutenin gene expressed in wheat, barley or maize endosperm. Any of these promoters may be used in the present invention.

Suitably the tissue-specific promoter may be a leaf-specific promoter. Suitable leaf-specific promoters may include ASYMMETRIC LEAVES 1 (AS1).

In another embodiment the promoter may be a developmentally-regulated promoter.

A developmentally-regulated promoter directs a change in the expression of a gene in one or more parts of a plant at a specific time during plant development. The gene may be expressed in that plant part at other times at a different (usually lower) level, and may also be expressed in other plant parts.

In one embodiment the promoter may be an inducible promoter.

An inducible promoter is capable of directing the expression of a gene in response to an inducer. In the absence of the inducer the gene will not be expressed. The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. A developmentally-regulated promoter might be described as a specific type of inducible promoter responding to an endogenous inducer produced by the plant or to an environmental stimulus at a particular point in the life cycle of the plant. Examples of known inducible promoters include those associated with wound response, such as described by Warner S A, Scott R, Draper J. (1993) (Isolation of an asparagus intracellular PR gene (AoPR1) wound-responsive promoter by the inverse polymerase chain reaction and its characterization in transgenic tobacco. Plant J. 3 191-201, which is incorporated herein by reference.), temperature response as disclosed by Benfey & Chua (1989) (Benfey, P. N., and Chua, N-H. (1989) Regulated genes in transgenic plants. Science 244 174-181, which is incorporated herein by reference), and chemically induced, as described by Gatz (1995) (Gatz, C. (1995) Novel inducible/repressible gene expression systems. Methods in Cell Biol. 50 411-424, which is incorporated herein by reference).

Thus in one embodiment the promoter may be selected from the group consisting of: the CERV promoter, the cauliflower mosaic virus 35S promoter (full or truncated), the rubisco promoter, the pea plastocyanin promoter, the nopaline synthase promoter, the chlorophyll r/b binding promoter, the high molecular weight glutenin promoter, the α, β-gliadin promoter, the hordein promoter and the patatin promoter.

The invention further provides a construct or vector comprising a nucleic acid encoding an EGY polypeptide operably linked with a leaf-specific promoter.

The invention also provides a construct or vector comprising a nucleic acid encoding an EGY polypeptide operably linked with a senescence-specific promoter.

The construct may be comprised in a vector. Suitably the vector may be a plasmid.

### Plant

The plant (or part thereof) or plant cell or plant propagation material according to the present invention may be a crop plant, e.g. a fruit crop, a seed crop, a legume or a nut crop.

The crop plant in accordance with the present invention may be selected from the group consisting of tobacco, tomato, strawberry, cherry, redcurrant, blackcurrant, gooseberry, raspberry, mulberry, peppers (Capsicum), peppers (Piper), water melon, melon, squash, gourd or aubergine (eggplant), olive, radish, horseradish, banana, apple, pears, peach, grape vine, citrus species, wheat, oat, barley, triticale, rice, quinoa (Chenopodium quinoa), fonio (Digitaria) maize, sorghum, rye, onion, leek, millet, buckwheat, sugarcane, sunflower, oilseed rapeseed (including canola), okra, coffee, and cocoa (Theobroma cacao), palm, cotton, coconut, sesame, safflower, flax, kapok, mustard, nutmeg, jojoba, peas, beans, alfalfa, lentils, soybeans, peanuts, almonds, pecans, pistachios, walnuts, Brazil nuts, hazelnuts, macadamia nuts, cashew nut, acorn, beechnuts, filbert nuts and chestnuts.

The plant (or part thereof) or plant cell or plant propagation material according to the present invention may be a fruit crop. A fruit crop in accordance with the present invention may be selected from the group consisting of: tomato, strawberry, cherry, redcurrant, blackcurrant, gooseberry, raspberry, mulberry, peppers (Capsicum), peppers (Piper), water melon, melon, squash, gourd, aubergine (eggplant), olive, radish, horseradish, banana, apple, pears, peach, grape vine and citrus species.

The plant (or part thereof) or plant cell or plant propagation material according to the present invention may be a seed crop. A seed crop in accordance with the present invention may be a cereal or grain crop, e.g. one selected from the group consisting of: wheat, oat, barley, triticale, rice, quinoa (Chenopodium quinoa), fonio (Digitaria), maize, sorghum, rye, onion, leek, millet, buckwheat, sugarcane. In another embodiment the seed crop in accordance with the present invention may be selected from the group consisting of: a sunflower, oilseed rapeseed (including canola), okra, coffee, cocoa (Theobroma cacao), palm, cotton, coconut, sesame, safflower, flax, kapok, mustard, nutmeg and jojoba.

The plant (or part thereof) or plant cell or plant propagation material according to the present invention may be a legume. A legume in accordance with the present invention may be selected from the group consisting of peas, beans, alfalfa, lentils, soybeans and peanuts.

The plant (or part thereof) or plant cell or plant propagation material according to the present invention may be a fruit or seed crop which is a nut crop. A nut crop in accordance with the present invention may be selected from the group consiting of almonds, pecans, pistachios, walnuts, Brazil nuts, hazelnuts, macadamia nuts, cashew nut, acorn, beechnuts, filbert nuts and chestnuts.

Preferably, the plant, plant cell or plant tissue, or host plant is a crop plant. By crop plant is meant any plant which is grown on a commercial scale for human or animal consumption or use for animal feed.

As used herein, the term ‘plant’ refers to any plant at any stage of its life cycle or development and its progenies. In general, unless otherwise specified, when referring to a “plant” it is intended to cover a plant at any stage of development, including single cells and seeds. Thus in particular embodiments, the present invention provides a plant cell, e.g. an isolated plant cell, having one or more characteristics of a “modified plant” as defined herein.

In one embodiment, the present invention provides a plant cell (e.g. a tobacco plant cell):
i) comprising an exogenous EGY gene (e.g. an exogenous polynucleotide) encoding an EGY protein as defined herein;
ii) comprising a construct or vector as defined herein; and/or
iii) obtainable (e.g obtained) by a method or use as described herein.

In another embodiment, the present invention provides a plant (e.g. a tobacco plant):
i) comprising an exogenous EGY gene (e.g. an exogenous polynucleotide) encoding an EGY protein as defined herein;
ii) which has been modified to achieve an increase in nitrogen-use efficiency and/or nitrogen-utilisation efficiency in comparison to an unmodified plant, wherein the modification is an increase in activity or expression of an EGY polypeptide in said modified plant;
iii) obtainable by a method or use as described herein;
iv) comprising a construct or vector as defined herein; and/or
v) comprising a cell as defined herein.

In one embodiment plant propagation material may be obtainable from a plant (e.g. a tobacco plant) of the invention.

The term “plant propagation material” as used herein refers to any plant matter taken from a plant from which further plants may be produced. Suitably the plant propagation material may be a seed.

In one embodiment the modified plant is a transgenic plant.

The term “consumed” as used herein means ingested by a human or animal (preferably human). The term “consumable” as used herein means being ingestable by a human or animal (preferably human). Ingestion may be in the form of eating, e.g. entering the body via the mouth for digestion and absorption, in which case the plants will be edible plants. In other embodiments the plants may be consumed or consumable by burning or heating the plant material or an extract (e.g. a tobacco extract) thereof and inhaling the fumes or smoke thus produced. In the latter case the consumption may be via the mouth and lungs.

The present invention further provides for harvested leaf of a plant in accordance with the present invention. Suitably the harvested leaf may be a harvested tobacco leaf.

The term harvested means that the leaf or leaves of the plant are removed from the roots of the plant. The harvested leaf may be comprised of leaf and stem material.

Suitably the harvested leaf (e.g. tobacco leaf) may be subjected to downstream processing. Thus in one embodiment the harvested leaf may be processed to produce a processed leaf.

In a particularly preferred embodiment, the plant (or part thereof) or plant cell or plant propagation material according to the present invention is a tobacco plant.

### Tobacco Plant

The present invention provides methods, uses directed to tobacco plants as well as a tobacco cell, a tobacco plant and a plant propagation material.

The term “tobacco plant” as used herein refers to a plant in the genus Nicotiana that is used in the production of tobacco products. Non-limiting examples of suitable tobacco plants include N. tabacum and N. rustica (for example, TN90, K326, LA B21, LN KY171, TI 1406, Basma, Galpao, Perique, Beinhart 1000-1, and Petico). It is not intended that the term “tobacco” extends to Nicotiana species that are not useful for the production of tobacco products.

Thus, in one embodiment a tobacco plant does include Nicotiana plumbaginifolia.

The tobacco material can be derived from varieties of Nicotiana tabacum species, commonly known as Burley varieties, flue or bright varieties, dark varieties and oriental/Turkish varieties. In some embodiments, the tobacco material is derived from a Burley, Virginia, flue-cured, air-cured, fire-cured, Oriental, or a dark tobacco plant. The tobacco plant may be selected from Maryland tobacco, rare tobacco, speciality tobacco, expanded tobacco or the like.

In one embodiment, the tobacco plant is not a Burley type.

The use of tobacco cultivars and elite tobacco cultivars is also contemplated herein. The tobacco plant for use herein may therefore be a tobacco variety or elite tobacco cultivar.

Particularly useful Nicotiana tabacum varieties include dark type, flue-cured type, and Oriental type tobaccos.

In some embodiments, the tobacco plant may be, for example, selected from one or more of the following varieties: N. tabacum AA 37-1, N. tabacum B 13P, N. tabacum Xanthi (Mitchell-Mor), N. tabacum KT D#3 Hybrid 107, N. tabacum Bel-W3, N. tabacum 79-615, N. tabacum Samsun Holmes NN, F4 from cross N. tabacum BU21 x N. tabacum Hoja Parado, line 97, N. tabacum KTRDC#2 Hybrid 49, N. tabacum KTRDC#4 Hybrid 1 10, N. tabacum Burley 21, N. tabacum PM016, N. tabacum KTRDC#5 KY 160 SI, N. tabacum KTRDC#7 FCA, N. tabacum KTRDC#6 TN 86 SI, N. tabacum PM021, N. tabacum K 149, N. tabacum K 326, N. tabacum K 346, N. tabacum K 358, N. tabacum K 394, N. tabacum K 399, N. tabacum K 730, N. tabacum KY 10, N. tabacum KY 14, N. tabacum KY 160, N. tabacum KY 17, N. tabacum KY 8959, N. tabacum KY 9, N. tabacum KY 907, N. tabacum MD 609, N. tabacum McNair 373, N. tabacum NC 2000, N. tabacum PG 01, N. tabacum PG 04, N. tabacum P01, N. tabacum P02, N. tabacum P03, N. tabacum RG 1 1, N. tabacum RG 17, N. tabacum RG 8, N. tabacum Speight G-28, N. tabacum TN 86, N. tabacum TN 90, N. tabacum VA 509, N. tabacum AS44, N. tabacum Banket A1, N. tabacum Basma Drama B84/31, N. tabacum Basma I Zichna ZP4/B, N. tabacum Basma Xanthi BX 2A, N. tabacum Batek, N. tabacum Besuki Jember, N. tabacum C104, N. tabacum Coker 319, N. tabacum Coker 347, N. tabacum Criollo Misionero, N. tabacum PM092, N. tabacum Delcrest, N. tabacum Djebel 81, N. tabacum DVH 405, N. tabacum Galpao Comum, N. tabacum HB04P, N. tabacum Hicks Broadleaf, N. tabacum Kabakulak Elassona, N. tabacum PM102, N. tabacum Kutsage E1, N. tabacum KY 14xL8, N. tabacum KY 171, N. tabacum LA BU 21, N. tabacum McNair 944, N. tabacum NC 2326, N. tabacum NC 71, N. tabacum NC 297, N. tabacum NC 3, N. tabacum PVH 03, N. tabacum PVH 09, N. tabacum PVH 19, N. tabacum PVH 21 10, N. tabacum Red Russian, N. tabacum Samsun, N. tabacum Saplak, N. tabacum Simmaba, N. tabacum Talgar 28, N. tabacum PM132, N. tabacum Wislica, N. tabacum Yayaldag, N. tabacum NC 4, N. tabacum TR Madole, N. tabacum Prilep HC-72, N. tabacum Prilep P23, N. tabacum Prilep PB 156/1, N. tabacum Prilep P12-2/1, N. tabacum Yaka JK-48, N. tabacum Yaka JB 125/3, N. tabacum T1-1068, N. tabacum KDH-960, N. tabacum TI-1070, N. tabacum TW136, N. tabacum PM204, N. tabacum PM205, N. tabacum Basma, N. tabacum TKF 4028, N. tabacum L8, N. tabacum TKF 2002, N. tabacum TN90, N. tabacum GR141, N. tabacum Basma xanthi, N. tabacum GR149, N. tabacum GR153, and N. tabacum Petit Havana.

Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF91 1, DT 538 LC Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KTY14xL8 LC, Little Crittenden, McNair 373, McNair 944, msKY 14xL8, Narrow Leaf Madole, Narrow Leaf Madole LC, NBH 98, N-126, N-777LC, N-7371 LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC 'Periq'e' tobacco, PVH03, PVH09, PVH19, PVH50, PVH51, R 610, R 630, R 7-1 1, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B 13P, Xanthi (Mitchell-Mor), Bel-W3, 79-615, Samsun Holmes NN, KTRDC number 2 Hybrid 49, Burley 21, KY 8959, KY 9, MD 609, PG 01, PG 04, P01, P02, P03, RG 1 1, RG 8, VA 509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpao Comum, HB04P, Hicks Broadleaf, Kabakulak Elassona, Kutsage E1, LA BU 21, NC 2326, NC 297, PVH 21 10, Red Russian, Samsun, Saplak, Simmaba, Talgar 28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

In one embodiment the plant propagation material may be obtainable from a tobacco plant of the invention.

A “plant propagation material” as used herein refers to any plant matter taken from a plant from which further plants may be produced.

Suitably the plant propagation material may be a seed.

In one embodiment the tobacco cell, tobacco plant and/or plant propagation material of the invention may comprise an exogenous EGY protein. In another embodiment the tobacco cell, tobacco plant and/or plant propagation material may comprise a construct or vector according to the invention. In another embodiment the tobacco cell, tobacco plant and/or plant propagation material may be obtainable (e.g. obtained) by a method according to the invention.

Suitably a tobacco plant according to the present invention may comprise a reduced amount of at least one tobacco-specific nitrosamine(s) when compared to an unmodified tobacco plant, wherein the modification is an increase in activity or expression of an EGY protein in said modified plant.

In one embodiment the tobacco plant in accordance with the present invention comprises a tobacco cell of the invention.

In another embodiment the plant propagation material may be obtainable (e.g. obtained) from a tobacco plant of the invention.

In another embodiment the tobacco cell, tobacco plant and/or plant propagation material may comprise an EGY polynucleotide or gene or an EGY polypeptide as defined herein.

Suitably, the tobacco cell, tobacco plant and/or plant propagation material may comprise:
i) a polynucleotide sequence shown herein as SEQ ID NO: 4, 5, 21 or 22 or a polynucleotide sequence which has at least 70% sequence identity to SEQ ID NO: 4, 5, 21 or 22, or
ii) a polynucleotide which encodes a polypeptide comprising the amino acid sequence shown herein as SEQ ID NO: 2 or SEQ ID NO: 3 or a functional fragment thereof or a sequence which has at least 70% sequence identity to SEQ ID NO: 2 or 3 or a functional fragment thereof
iii) a polynucleotide sequence which can hybridize to the polynucleotide taught in i), or ii) above under high stringency conditions, or
iv) a polynucleotide sequence which has at least 70% (preferably 85%, more preferably 90%) identity with the polynucleotide shown in i), ii) or iii) above, or v) a polynucleotide sequence which differs from polynucleotide shown in i), ii) or iii) due to degeneracy of the genetic code.

In one embodiment the polynucleotide sequence may have at least 80% identity with SEQ ID NO: 4, 5, 21 or 22. In one embodiment the polynucleotide sequence may have at least 80% identity with SEQ ID NO: 4 or 5.

Suitably the polynucleotide sequence may have at least 90% identity with SEQ ID NO: 4, 5, 21 or 22. In one embodiment the polynucleotide sequence may have at least 90% identity with SEQ ID NO: 4 or 5.

Suitably the polynucleotide sequence may have at least 95% identity (more suitably at least 99% identity) with SEQ ID NO: 4, 5, 21 or 22. In one embodiment the polynucleotide sequence may have at least 95% identity with SEQ ID NO: 4 or 5.

In another embodiment the tobacco cell, tobacco plant and/or plant propagation material may comprise an EGY protein comprising a polypeptide sequence which has the following conserved residues: a GNLR motif comprising Gly-176, Asn-177, Leu-178 and Arg-179; a HEXXH motif comprising His-311, Glu-312, X-313, X-314 and His-311; and a NXXPXXXLDG motif comprising Asn-441, X-442, X-443, Pro-444, X-445, X-446, X-447, Leu-448, Asp-449 and Gly-450; wherein X is any amino acid and wherein the numbering of the conserved residues means the effective equivalent position with the EGY protein is aligned with the Arabidopsis thaliana EGY1 protein having SEQ ID NO: 1.

Suitably the polypeptide may comprise a polypeptide shown as SEQ ID NO: 2 or SEQ ID NO: 3 or a functional fragment thereof or a sequence which has at least 70% sequence identity to SEQ ID NO: 2 or SEQ ID NO: 3 or a functional fragment thereof.

Suitably the polypeptide may have at least 80% identity with SEQ ID NO: 2 or SEQ ID NO: 3 or a functional fragment thereof. More suitably the polypeptide may have at least 90% identity with

SEQ ID NO: 2 or SEQ ID NO: 3 or a functional fragment thereof. In particular the polypeptide may have at least 95% identity with SEQ ID NO: 2 or SEQ ID NO: 3 or a functional fragment thereof.

Suitably the polypeptide may have at least 80% identity with SEQ ID NO: 2 or SEQ ID NO: 3. More suitably the polypeptide may have at least 90% identity with SEQ ID NO: 2 or SEQ ID NO: 3. In particular the polypeptide may have at least 95% identity with SEQ ID NO: 2 or SEQ ID NO: 3.

Most suitably the polypeptide may have at least 99% identity with SEQ ID NO: 2 or SEQ ID NO: 3.

In one embodiment there is provided the use of a tobacco cell as provided for in the foregoing embodiments for production of a tobacco product.

Additionally there is provided the use of a tobacco plant as described herein to breed a tobacco plant.

The present invention also provides in another embodiment the use of a tobacco plant of the foregoing embodiments for the production of a tobacco product.

In another embodiment there is provided the use of a tobacco plant of the invention to grow a crop.

### Commercially Desirable Traits

The term “commercially desirable traits” will include traits such as yield and/or quality.

### Molecular Marker Assisted Selection

The molecular marker assisted selection may comprise performing PCR to identify a desired naturally-occurring genetic variant or an introgressed nucleic acid sequence comprising a variant EGY nucleotide sequence with altered expression or activity of an EGY protein.

In one embodiment the variant EGY nucleotide sequence may increase the expression or activity of an EGY protein

### Products

The present invention also provides for products obtainable or obtained from a plant (e.g. a tobacco plant) according to the present invention.

In one embodiment there is provided the use of a tobacco plant of the invention to produce a tobacco leaf.

Suitably the tobacco leaf may be subjected to downstream applications such as processing. Thus in one embodiment the use of the foregoing embodiment may provide a processed tobacco leaf. Suitably the tobacco leaf may be subjected to curing, fermenting, pasteurising or combinations thereof.

In another embodiment the tobacco leaf may be cut. In some embodiments the tobacco leaf may be cut before or after being subjected to curing, fermenting, pasteurising or combinations thereof.

In one embodiment the present invention provides a harvested leaf of a tobacco plant of the invention.

In a further embodiment the harvested leaf may be obtainable (e.g. obtained) from a tobacco plant propagated from a propagation material of the present invention.

In another embodiment there is provided a harvest leaf obtainable from a method or use of the present invention.

Suitably the harvested leaf may be a cut harvested leaf.

In some embodiments the harvested leaf may comprise viable tobacco cells. In other embodiments the harvested leaf may be subjected to further processing.

There is also provided a processed tobacco leaf.

The processed tobacco leaf may be obtainable from a tobacco plant of the invention. Suitably the processed tobacco leaf may be obtainable from a tobacco plant obtained in accordance with any of the methods and/or uses of the present invention.

In another embodiment the processed tobacco leaf may be obtainable from a tobacco plant propagated form a tobacco plant propagation material according to the present invention.

The processed tobacco leaf of the present invention may be obtainable by processing a harvested leaf of the invention.

The term “processed tobacco leaf” as used herein refers to a tobacco leaf that has undergone one or more processing steps to which tobacco is subjected to in the art. A “processed tobacco leaf” comprises no or substantially no viable cells.

The term “viable cells” refers to cells which are able to grow and/or are metabolically active. Thus, if a cell is said to not be viable, also referred to as “non-viable” then a cell does not display the characteristics of a viable cell.

The term “substantially no viable cells” means that less than about 5% of the total cells are viable. Preferably, less than about 3%, more preferably less than about 1%, even more preferably less than about 0.1% of the total cells are viable.

In one embodiment the processed tobacco leaf may be processed by one or more of: curing, fermenting and/or pasteurising.

Suitably the processed tobacco leaf may be processed by curing.

Tobacco leaf may be cured by any method known in the art. In one embodiment tobacco leaf may be cured by one or more of the curing methods selected from the group consisting of: air curing, fire curing, flue curing and sun curing.

Suitably the tobacco leaf may be air cured.

Typically air curing is achieved by hanging tobacco leaf in well-ventilated barns and allowing to dry. This is usually carried out over a period of four to eight weeks. Air curing is especially suitable for burley tobacco.

Suitably the tobacco leaf may be fire cured. Fire curing is typically achieved by hanging tobacco leaf in large barns where fires of hardwoods are kept on continuous or intermittent low smoulder and usually takes between three days and ten weeks, depending on the process and the tobacco.

In another embodiment the tobacco leaf may be flue cured. Flue curing may comprise stringing tobacco leaves onto tobacco sticks and hanging them from tier-poles in curing barns. The barns usually have a flue which runs from externally fed fire boxes. Typically this results in tobacco that has been heat-cured without being exposed to smoke. Usually the temperature will be raised slowly over the course of the curing with the whole process taking approximately 1 week.

Suitably the tobacco leaf may be sun cured. This method typically involves exposure of uncovered tobacco to the sun.

Suitably the processed tobacco leaf may be processed by fermenting.

Fermentation can be carried out in any manner known in the art. Typically during fermentation, the tobacco leaves are piled into stacks (a bulk) of cured tobacco covered in e.g. burlap to retain moisture. The combination of the remaining water inside the leaf and the weight of the tobacco generates a natural heat which ripens the tobacco. The temperature in the centre of the bulk is monitored daily. In some methods every week, the entire bulk is opened. The leaves are then removed to be shaken and moistened and the bulk is rotated so that the inside leaves go outside and the bottom leaves are placed on the top of the bulk. This ensures even fermentation throughout the bulk. The additional moisture on the leaves, plus the actual rotation of the leaves themselves, generates heat, releasing the tobacco's natural ammonia and reducing nicotine, while also deepening the colour and improving the tobacco's aroma. Typically the fermentation process continues for up to 6 months, depending on the variety of tobacco, stalk position on the leaf, thickness and intended use of leaf.

Suitably the processed tobacco leaf may be processed by pasteurising. Pasteurising may be particularly preferred when the tobacco leaf will be used to make a smokeless tobacco product, most preferably snus.

Tobacco leaf pasteurisation may be carried out by any method known in the art. For example pasteurisation may be carried out as detailed in J Foulds, L Ramstrom, M Burke, K Fagerstrom. Effect of smokeless tobacco (snus) on smoking and public health in Sweden (incorporated herein by reference).

Tobacco Control (2003) 12: 349-359, the teaching of which is incorporated herein by reference.

During the production of snus pasteurisation is typically carried out by a process in which the tobacco is heat treated with steam for 24-36 hours (reaching temperatures of approximately 100° C.). This results in an almost sterile product and without wishing to be bound by theory one of the consequences of this is believed to be a limitation of further TSNA formation.

In one embodiment the pasteurisation may be steam pasteurisation.

In some embodiments the processed tobacco leaf may be cut. The processed tobacco leaf may be cut before or after processing. Suitably, the processed tobacco leaf may be cut after processing.

In some embodiments the tobacco plant, harvested leaf of a tobacco plant and/or processed tobacco leaf may be used to extract nicotine. The extraction of nicotine can be achieved using any method known in the art. For example a method for extracting nicotine from tobacco is taught in U.S. Pat. No. 2,162,738 which is incorporated herein by reference.

In another aspect the present invention provides a tobacco product.

In one embodiment the tobacco product may be prepared from a tobacco plant of the invention or a part thereof.

Suitably the tobacco plant or part thereof may be propagated from a tobacco plant propagation material according to the present invention.

The term “part thereof” as used herein in the context of a tobacco plant refers to a portion of the tobacco plant. Preferably the “part thereof” is a leaf of a tobacco plant.

In another embodiment the tobacco product may be prepared from a harvested leaf of the invention.

In a further embodiment the tobacco product may be prepared from a processed tobacco leaf of the invention.

Suitably the tobacco product may be prepared from a tobacco leaf processed by one or more of: curing, fermenting and/or pasteurising.

Suitably the tobacco product may comprise a cut tobacco leaf, optionally processed as per the foregoing embodiment.

In one embodiment the tobacco product may be a smoking article.

As used herein, the term “smoking article” can include smokeable products, such as rolling tobacco, cigarettes, cigars and cigarillos whether based on tobacco, tobacco derivatives, expanded tobacco, reconstituted tobacco or tobacco substitutes.

In another embodiment the tobacco product may be a smokeless tobacco product.

The term “smokeless tobacco product” as used herein refers to a tobacco product that is not intended to be smoked and/or subjected to combustion. In one embodiment a smokeless tobacco product may include snus, snuff, chewing tobacco or the like.

In a further embodiment the tobacco product may be a tobacco heating device.

Typically in heated smoking articles, an aerosol is generated by the transfer of heat from a heat source to a physically separate aerosol-forming substrate or material, which may be located within, around or downstream of the heat source. During smoking, volatile compounds are released from the aerosol-forming substrate by heat transfer from the heat source and entrained in air drawn through the smoking article. As the released compounds cool, they condense to form an aerosol that is inhaled by the user.

Aerosol-generating articles and devices for consuming or smoking tobacco heating devices are known in the art. They can include, for example, electrically heated aerosol-generating devices in which an aerosol is generated by the transfer of heat from one or more electrical heating elements of the aerosol-generating device to the aerosol-forming substrate of a tobacco heating device.

Suitably the tobacco heating device may be an aerosol-generating device.

Preferably the tobacco heating device may be a heat-not-burn device. Heat-not-burn devices are known in the art and release compounds by heating, but not burning, tobacco.

An example of a suitable, heat-not-burn device may be one taught in WO2013/034459 or GB2515502 which are incorporated herein by reference.

In one embodiment the aerosol-forming substrate of a tobacco heating device may be a tobacco product in accordance with the present invention.

### Polynucleotides/Polypeptides/Constructs/Methods

In certain embodiments of the present invention, chimeric genes encoding a protein of interest (e.g. an EGY protein) may be transformed into plant cells leading to controlled expression of the protein of interest under the direction of a promoter. The promoters may be obtained from different sources including animals, plants, fungi, bacteria, and viruses. Promoters may also be constructed synthetically.

Exogenous genes may be introduced into plants according to the present invention by means of suitable vector, e.g. plant transformation vectors. A plant transformation vector may comprise an expression cassette comprising 5′-3′ in the direction of transcription, a promoter sequence, a gene of interest (e.g. a deregulated nitrate reductase) coding sequence, optionally including introns, and, optionally a 3′ untranslated, terminator sequence including a stop signal for RNA polymerase and a polyadenylation signal for polyadenylase. The promoter sequence may be present in one or more copies, and such copies may be identical or variants of a promoter sequence as described above. The terminator sequence may be obtained from plant, bacterial or viral genes. Suitable terminator sequences are the pea rbcS E9 terminator sequence, the nos terminator sequence derived from the nopaline synthase gene of Agrobacterium tumefaciens and the 35S terminator sequence from cauliflower mosaic virus, for example. A person skilled in the art will be readily aware of other suitable terminator sequences.

The expression cassette may also comprise a gene expression enhancing mechanism to increase the strength of the promoter. An example of such an enhancer element is one derived from a portion of the promoter of the pea plastocyanin gene, and which is the subject of International patent Application No. WO 97/20056 (incorporated herein by reference). Suitable enhancer elements may be the nos enhancer element derived from the nopaline synthase gene of Agrobacterium tumefaciens and the 35S enhancer element from cauliflower mosaic virus, for example. These regulatory regions may be derived from the same gene as the promoter DNA sequence or may be derived from different genes, from Nicotiana tabacum or other organisms, for example from a plant of the family Solanaceae, or from the subfamily Cestroideae. All of the regulatory regions should be capable of operating in cells of the tissue to be transformed.

The promoter DNA sequence may be derived from the same gene as the gene of interest (e.g. the gene the promoter is going to direct, for instance a gene encoding a the modification of a plant to increase the activity or expression of a LBD nitrogen-responsive transcription factor) coding sequence used in the present invention or may be derived from a different gene, from Nicotiana tabacum, or another organism, for example from a plant of the family Solanaceae, or from the subfamily Cestroideae. When referring to a “chimeric gene”, it is meant that the nucleic acid sequence encoding a gene of interest (e.g. a gene encoding a deregulated nitrate reductase) is derived from a different origin (e.g. from a different gene, or from a different species) to the promoter sequence which directs its expression.

The expression cassette may be incorporated into a basic plant transformation vector, such as pBIN 19 Plus, pBI 101, or other suitable plant transformation vectors known in the art. In addition to the expression cassette, the plant transformation vector will contain such sequences as are necessary for the transformation process. These may include the Agrobacterium vir genes, one or more T-DNA border sequences, and a selectable marker or other means of identifying transgenic plant cells.

The term “plant transformation vector” means a construct capable of in vivo or in vitro expression. Preferably, the expression vector is incorporated in the genome of the organism. The term “incorporated” preferably covers stable incorporation into the genome.

Techniques for transforming plants are well known within the art and include Agrobacterium-mediated transformation, for example. The basic principle in the construction of genetically modified plants is to insert genetic information in the plant genome so as to obtain a stable maintenance of the inserted genetic material. A review of the general techniques may be found in articles by Potrykus (Annu Rev Plant Physiol Plant Mol Biol [1991] 42:205-225) and Christon (AgroFood-Industry Hi-Tech March/April 1994 17-27), each of which are incorporated herein by reference.

Typically, in Agrobacterium-mediated transformation a binary vector carrying a foreign DNA of interest, i.e. a chimeric gene, is transferred from an appropriate Agrobacterium strain to a target plant by the co-cultivation of the Agrobacterium with explants from the target plant. Transformed plant tissue is then regenerated on selection media, which selection media comprises a selectable marker and plant growth hormones. An alternative is the floral dip method (Clough & Bent, 1998) whereby floral buds of an intact plant are brought into contact with a suspension of the Agrobacterium strain containing the chimeric gene, and following seed set, transformed individuals are germinated and identified by growth on selective media. Direct infection of plant tissues by Agrobacterium is a simple technique which has been widely employed and which is described in Butcher D. N. et al., (1980), Tissue Culture Methods for Plant Pathologists, eds.: D. S. Ingrams and J. P. Helgeson, 203-208, which is incorporated herein by reference.

Further suitable transformation methods include direct gene transfer into protoplasts using polyethylene glycol or electroporation techniques, particle bombardment, micro-injection and the use of silicon carbide fibres for example.

Transforming plants using ballistic transformation, including the silicon carbide whisker technique are taught in Frame B R, Drayton P R, Bagnaall S V, Lewnau C J, Bullock W P, Wilson H M, Dunwell J M, Thompson J A & Wang K (1994). Production of fertile transgenic maize plants by silicon carbide whisker-mediated transformation is taught in The Plant Journal 6: 941-948) and viral transformation techniques is taught in for example Meyer P, Heidmmm I & Niedenhof I (1992). The use of cassava mosaic virus as a vector system for plants is taught in Gene 110: 213-217. Further teachings on plant transformation may be found in EP-A-0449375 (each of which are incorporated herein by reference).

In a further aspect, the present invention relates to a vector system which carries a nucleotide sequence encoding a gene of interest (e.g. a gene encoding an EGY protein) and introducing it into the genome of an organism, such as a plant. The vector system may comprise one vector, but it may comprise two vectors. In the case of two vectors, the vector system is normally referred to as a binary vector system. Binary vector systems are described in further detail in Gynheung Anetal, (1980), Binary Vectors, Plant Molecular Biology Manual A3, 1-19, incorporated herein by reference.

One extensively employed system for transformation of plant cells uses the Ti plasmid from Agrobacterium tumefaciens or a Ri plasmid from Agrobacterium rhizogenes Anetal., (1986), Plant Physiol. 81, 301-305 and Butcher D. N. et al., (1980), Tissue Culture Methods for Plant Pathologists, eds.: D. S. Ingrams and J. P. Helgeson, 203-208 (each of which are incorporated herein by reference). After each introduction method of the desired exogenous gene according to the present invention in the plants, the presence and/or insertion of further DNA sequences may be necessary. The use of T-DNA for the transformation of plant cells has been intensively studied and is described in EP-A-120516; Hoekema, in: The Binary Plant Vector System Offset-drukkerij Kanters B. B., Amsterdam, 1985, Chapter V; Fraley, etal., Crit. Rev. Plant Sci., 4:1-46; and Anetal., EMBO J (1985) 4:277-284 (each of which are incorporated herein by reference).

Plant cells transformed with an exogenous gene encoding a protein of interest (e.g. an EGY protein) may be grown and maintained in accordance with well-known tissue culturing methods such as by culturing the cells in a suitable culture medium supplied with the necessary growth factors such as amino acids, plant hormones, vitamins, etc.

The term “transgenic plant” in relation to the present invention includes any plant that comprises an exogenous gene encoding a gene of interest, e.g. a gene encoding an EGY protein, according to the present invention. Preferably the exogenous gene is incorporated in the genome of the plant.

The terms “transgenic plant” and “chimeric gene” do not cover native nucleotide coding sequences in their natural environment when they are under the control of their native promoter which is also in its natural environment.

In one aspect, a nucleic acid sequence, chimeric gene, plant transformation vector or plant cell according to the present invention is in an isolated form. The term “isolated” means that the sequence is at least substantially free from at least one other component with which the sequence is naturally associated in nature and as found in nature.

In one aspect, a nucleic acid sequence, chimeric gene, plant transformation vector or plant cell according to the invention is in a purified form. The term “purified” means in a relatively pure state, e.g. at least about 90% pure, or at least about 95% pure or at least about 98% pure.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232—incorporated herein by reference).

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805—incorporated herein by reference. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491)—incorporated herein by reference.

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared and/or isolated from a suitable source, or it may be made synthetically or it may be prepared by use of recombinant DNA techniques.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence and/or fragments should provide and/or encode a polypeptide which retains the functional activity and/or enhances the activity of the enzyme.

Typically, the homologous sequences will comprise the same active sites etc. as the subject amino acid sequence for instance or will encode the same active sites. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Homology or identity comparisons can be conducted by eye, or more usually, with the aid of readily available sequence comparison programs. These commercially available computer programs can calculate % homology between two or more sequences.

% homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244, incorporated herein by reference).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 0 |
| GAP EXTENSION | 0 |

| FOR CLUSTAL | DNA | PROTEIN | |
|---|---|---|---|
| WORD SIZE | 2 | 1 | K triple |
| GAP PENALTY | 15 | 10 | |
| GAP EXTENSION | 6.66 | 0.1 | |

In one embodiment, CLUSTAL may be used with the gap penalty and gap extension set as defined above.

In some embodiments the gap penalties used for BLAST or CLUSTAL alignment may be different to those detailed above. The skilled person will appreciate that the standard parameters for performing BLAST and CLUSTAL alignments may change periodically and will be able to select appropriate parameters based on the standard parameters detailed for BLAST or CLUSTAL alignment algorithms at the time.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence may be determined over the whole sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, ß-alanine*, L-α-amino butyric acid*, L-γ-amino butyric acid*, L-α-amino isobutyric acid*, L-ε-amino caproic acid#, 7-amino heptanoic acid*, L-methionine sulfone^{#}*, L-norleucine*, L-norvaline*, p-nitro-L-phenylalanine*, L-hydroxproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid* and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134—incorporated herein by reference

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also relates to nucleotide sequences that can hybridise to the nucleotide sequences of the present invention (including complementary sequences of those presented herein).

Preferably, hybridisation is determined under stringent conditions (e.g. 50° C. and 0.2×SSC {1×SSC=0.15 M NaCl, 0.015 M Na₃citrate pH 7.0}).

More preferably, hybridisation is determined under high stringent conditions (e.g. 65° C. and 0.1×SSC {1×SSC=0.15 M NaCl, 0.015 M Na₃citrate pH 7.0}).

In one aspect the sequence for use in the present invention is a synthetic sequence—i.e. a sequence that has been prepared by in vitro chemical or enzymatic synthesis. It includes, but is not limited to, sequences made with optimal codon usage for host organisms.

The term “expression vector” means a construct capable of in vivo or in vitro expression.

Preferably, the expression vector is incorporated into the genome of a suitable host organism. The term “incorporated” preferably covers stable incorporation into the genome.

The nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described herein to provide for expression of a polypeptide of the present invention.

The choice of vector e.g. a plasmid, cosmid, or phage vector will often depend on the host cell into which it is to be introduced.

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27)—each of which are incorporated herein by reference. Further teachings on plant transformation may be found in EP-A-0449375.

Amino acids are referred to herein using the name of the amino acid, the three letter abbreviation or the single letter abbreviation.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and equivalents thereof known to those skilled in the art, and so forth.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Advantages

The key advantages associated with the present invention are an improvement in NUE and NUTL in a plant which has been modified to increase the expression or activity of an EGY protein. Such an advantage may allow an increased biomass in harvestable product or improved growth of the modified plant in nitrogen deficient conditions.

A further particular advantage associated with the present invention is the reduction of tobacco-specific nitrosamine concentration (e.g. NNK and/or NNN) in a tobacco product prepared from a tobacco plant which has been modified to increase the activity or expression of an EGY protein.

The invention will now be described, by way of example only, with reference to the following Figures and Examples.

### EXAMPLES

### Example 1—Identification of Yellow Burley 1 (Yb1) and Yellow Burley 2 (Yb2) Loci

Compared to tobacco cultivars of other market classes, burley tobacco has a high degree of chlorophyll deficiency that is most evident on the stems, stalks, and leaf midveins of burley tobacco plants and that becomes more pronounced with plant age. In addition, burley tobacco has increased alkaloid levels, reduced nitrogen use efficiency, reduced nitrogen utilization efficiency, increased leaf nitrate nitrogen (NO₃—N) and increased tobacco specific nitrosamines (TSNAs)—in particular compared to flue-cured tobacco. The chlorophyll-deficient phenotype of burley tobacco is conferred by a double homozygous recessive genotype (yb1yb1 yb2yb2) at the Yellow Burley 1 (Yb1) and Yellow Burley 2 (Yb2) loci, which have been reported to reside on chromosome B and chromosome O, respectively. Only a single dominant allele at either of the two loci is required to establish the normal, more-green phenotype.

### Fine Mapping of Yb1 and Yb2

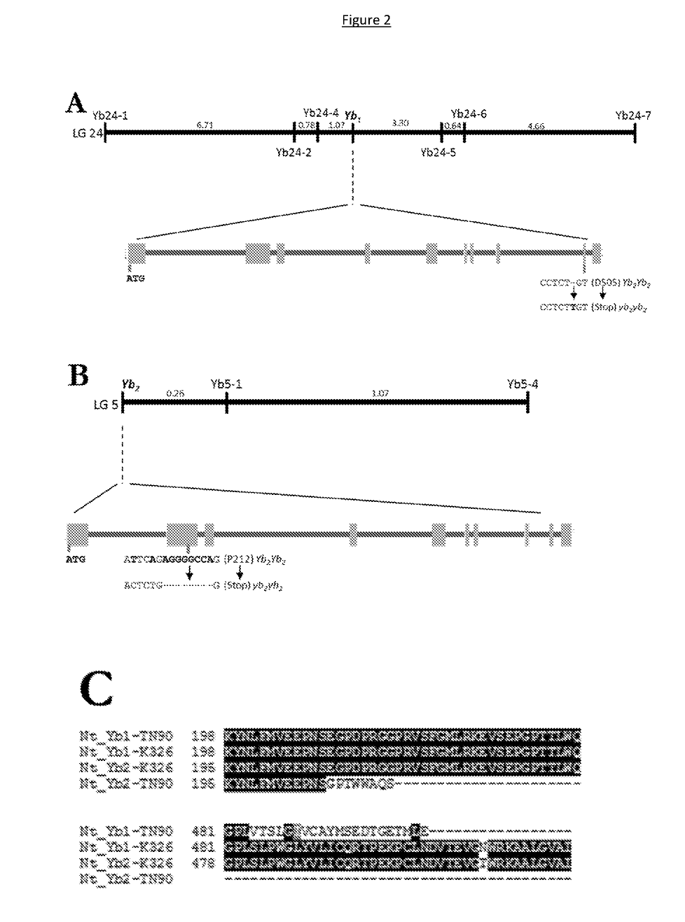

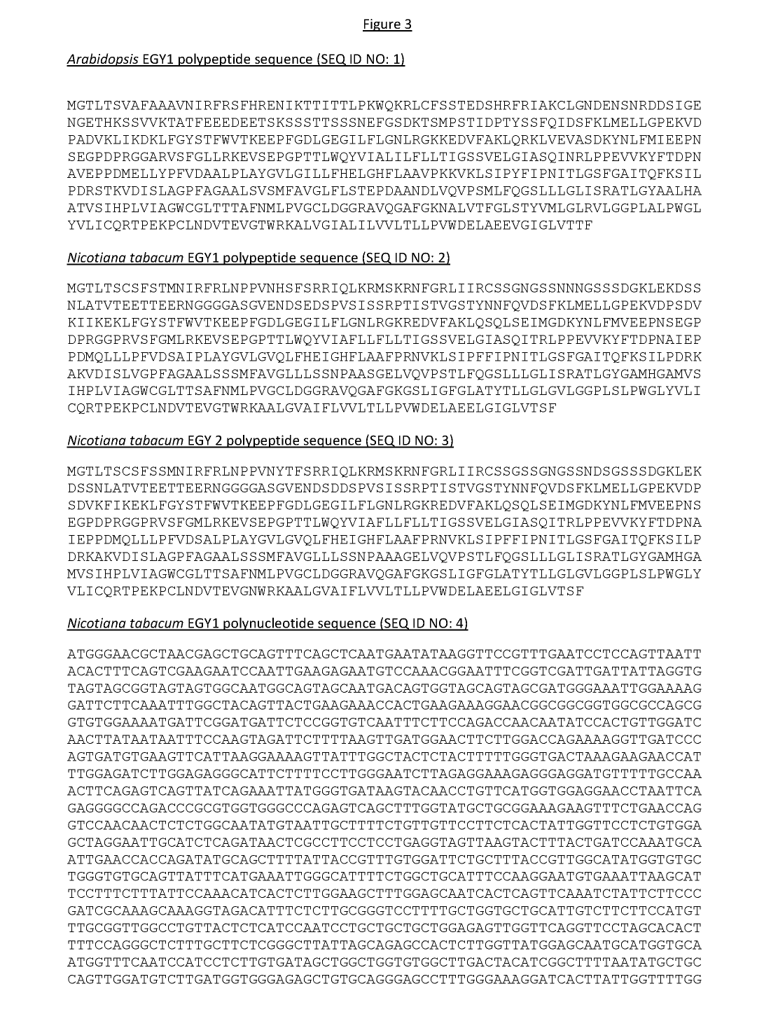

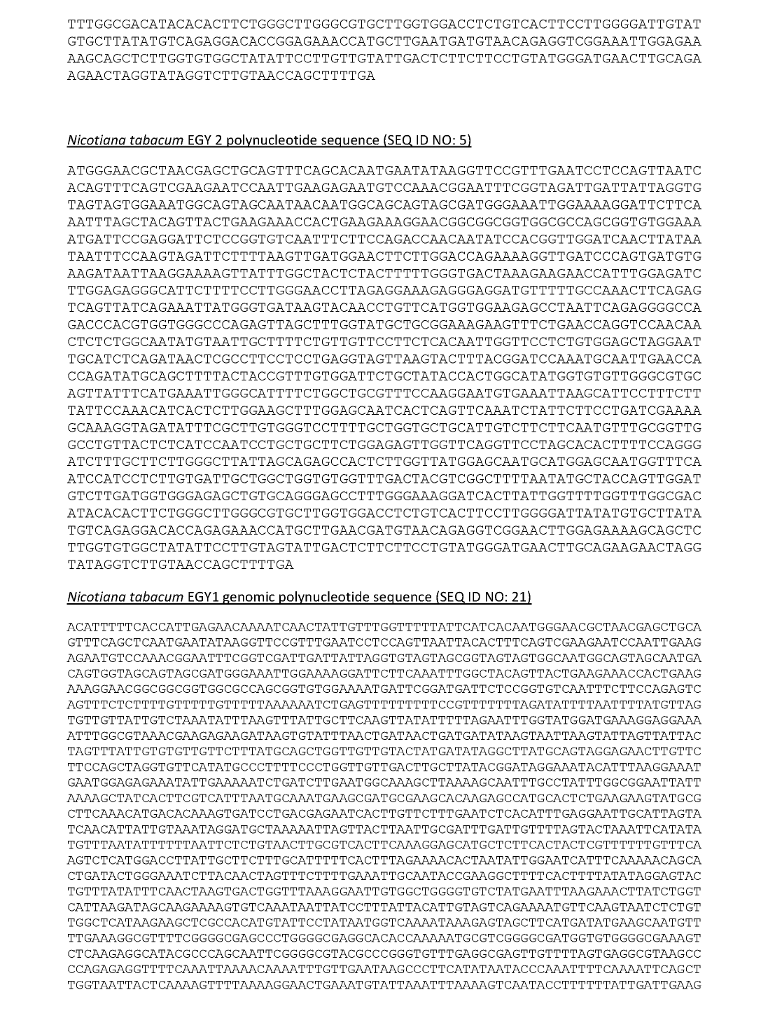

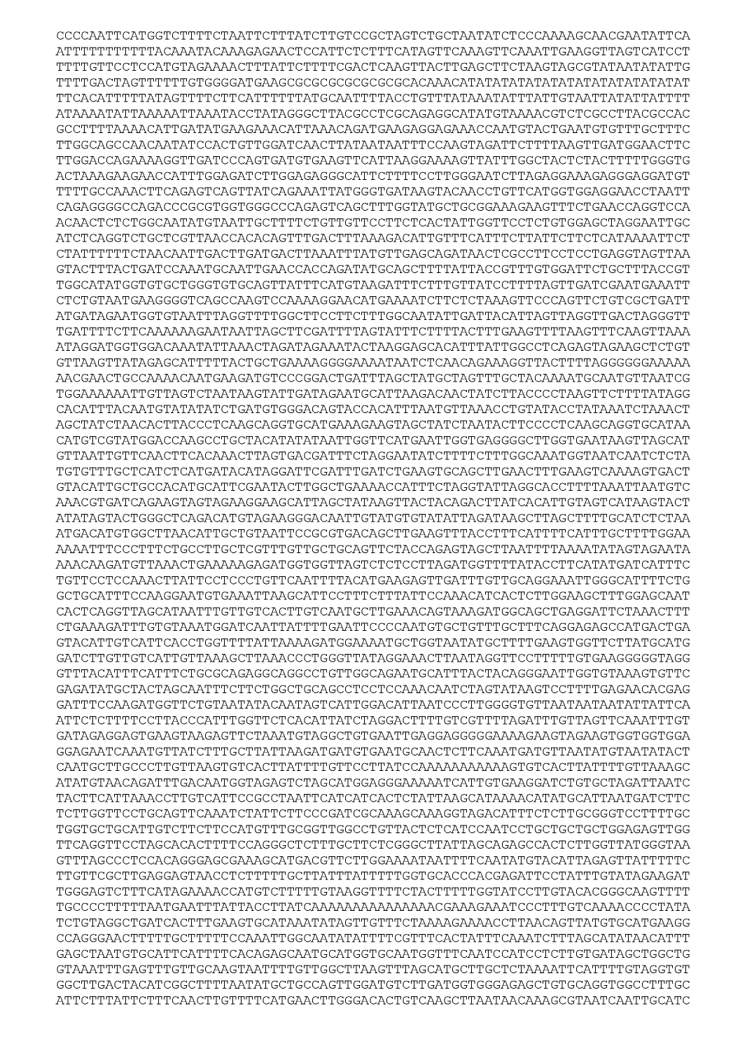

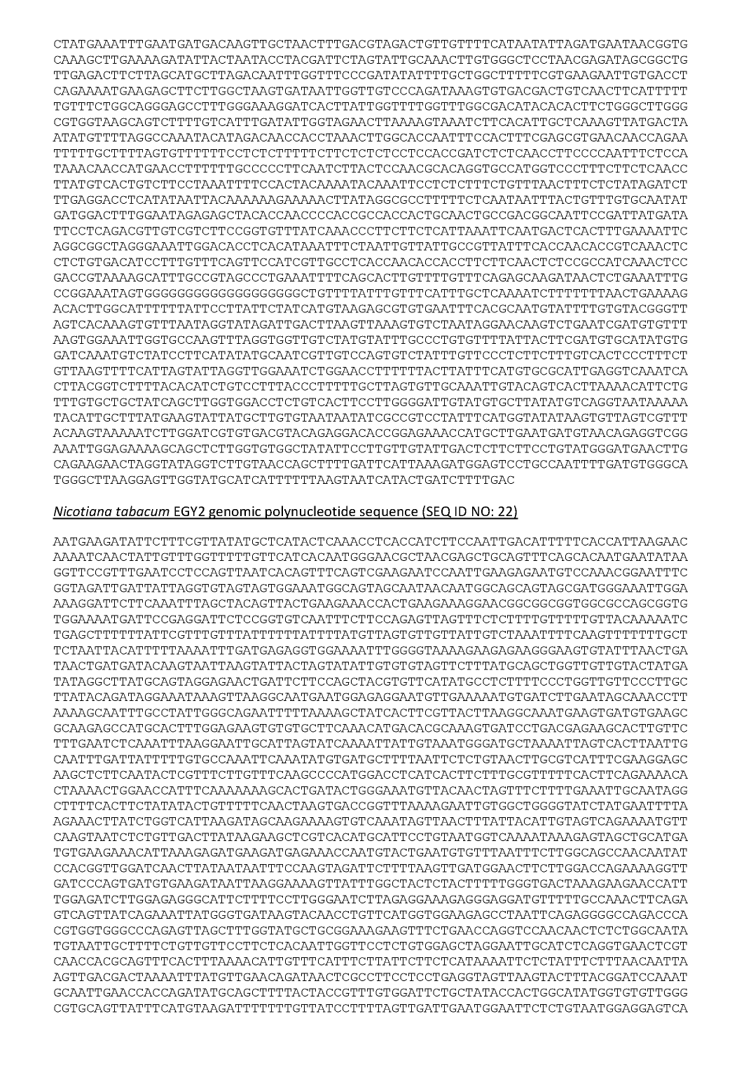

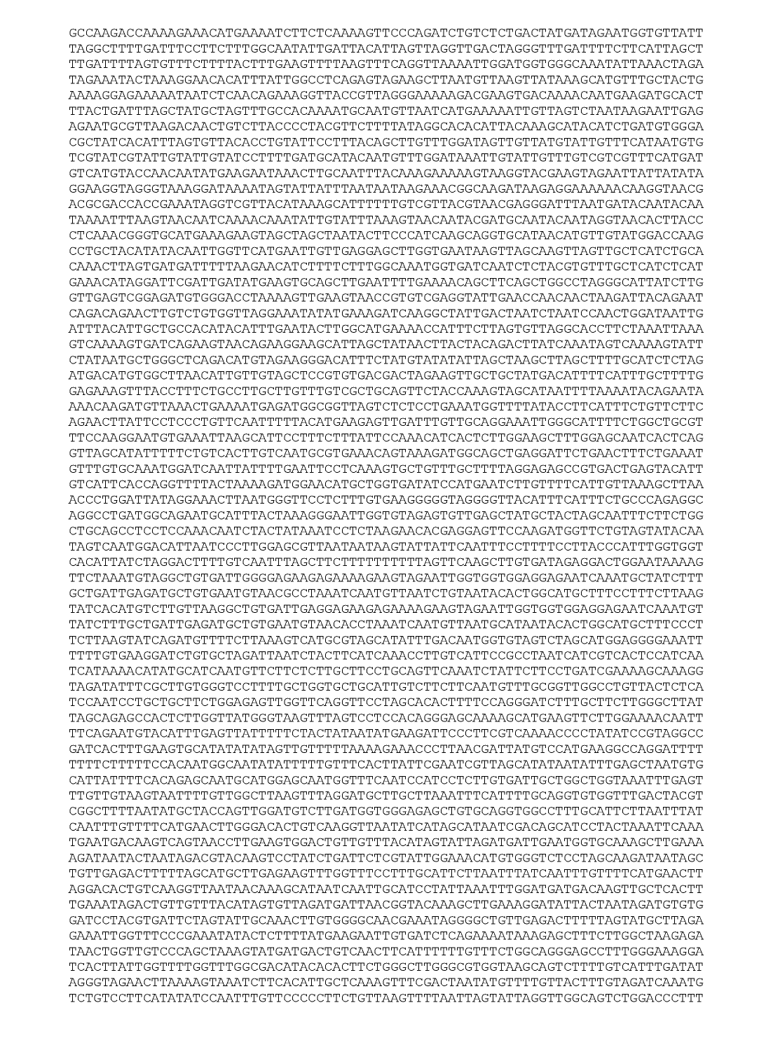

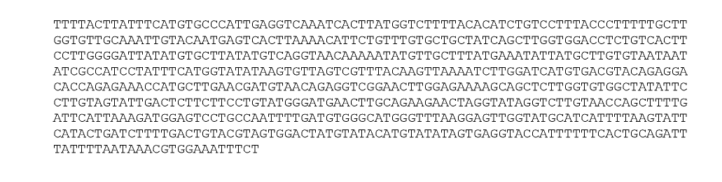

Three pairs of lines isogenic for alleles at the Yb₁ and Yb₂ loci were genotyped using a 30 K Infinium SNP chip. Two genomic regions were found to exhibit polymorphisms for all three pairs of nearly isogenic lines. Through co-segregation analyses with microsatellite markers on the high density microsatellite marker linkage map of Bindler et al. (2011; Theor Appl Genet; 123: 219-230—incorporated herein by reference), these SNP markers were found to reside on N. tabacum linkage groups (LG) 5 and 24, which were donated by progenitor species N. sylvestris and N. tomentosiformis, respectively. Two SNP markers estimated to span a region of 1.33 cM on LG 5, and six SNP markers estimated to cover a region of 17.16 cM on LG 24, were converted to KASP markers (FIG. 2) for fine mapping the Yb₁ and Yb₂ loci using the BWDH8/NC1426-17//NC1426-17 and BWDH16/NC1426-17//NC1426-17 BC₁F₁ populations which were expected to segregate for the wild-type allele at one Yb locus and to be fixed for the recessive allele at the alternative Yb locus. Genotyping of the parental lines of the two mapping populations (BWDH8, BWDH16, and NC1426-7) with the eight KASP markers was used to infer that the BWDH8-derived population was segregating for the markers on LG 5 and the BWDH16-derived population was segregating for the markers on LG 24.

Screening of 384 BC₁F₁ individuals in the BWDH8/NC1426-17//NC1426-17 BC₁F₁ population with the two selected LG 5 KASP markers refined the position of one Yb loci to 0.26 cM from SNP marker Yb5-1 (FIG. 2). Screening of 384 BC₁F₁ individuals in the BWDH16/NC1426-17//NC1426-17 BC, F₁ population with the six selected LG 24 KASP markers refined the position of the other Yb marker to a 4.37 interval between SNP markers Yb24-4 and Yb24-5, and 1.07 cM away from marker Yb24-4 (FIG. 2). To identify scaffolds with potential candidate genes, SNP marker sequences most closely associated to either Yb locus were aligned to scaffolds from a draft N. tabacum genome sequence. Three scaffolds (3,876,640 bp) with a total of 79 predicted genes were identified for LG 5 and three scaffolds (8,458,802 bp) with a total of 223 predicted genes were identified for LG 24.

Sequence similarity searches to the Arabidopsis genome assembly (The Arabidopsis Information Resource, TAIR) (www.arabidopsis.org) were carried out to determine the potential function of predicted genes. Genes predicted to be associated with nitrogen assimilation, nitrogen use physiology, or chloroplast activities were considered as potential candidate genes underlying the yellow burley phenotype. Two such candidates were identified for the genomic region of interest on LG 5 and four for the region of interest on LG 24. Full length Arabidopsis cDNA sequences corresponding to the six genes of interest were obtained from TAIR and BLAST searched against genome sequence data for K326 (Yb₁ Yb₁ Yb₂ Yb₂) and TN90 (yb₁yb₁ yb₂yb₂) (GCA_00715075.1 and GCA_00715135.1, respectively; www.ncbi.nlm.nih.gov). No differences were found between the K326 and TN90 draft sequences for N. tabacum homologs of the Arabidopsis genes CJD1, FtsZ2-1, ChlAKR, or RCA. In the CRTISO homolog, an 80 bp deletion was identified for TN90 between the seventh and eighth exon, but this did not result in predicted changes in the amino acid sequence. In the EGY1 homolog, however, 8 bp and 111 bp deletions were identified in TN90 relative to K326, with the former predicted to lead to alternative splicing and a predicted extra exon.

The EGY1 (ethylene-dependent gravitropism-deficient and yellow green protein 1) homolog (hereafter referred to as NtEGY1) was investigated further because of the predicted dramatic amino acid sequence differences between TN90 and K326. Considering the allotretraploid nature of N. tabacum, where the S and T genomes were contributed by N. sylvestris and N. tomentosiformis, respectively, the NtEGY1 sequence was BLAST searched against the N. tabacum genome sequence to identify potential highly similar sequences. A predicted gene with an 89 percent nucleotide identity was identified as a potential homeolog, and tentatively designated as NtEGY2. BLAST comparisons of the NtEGY2 sequence from K326 and TN 90LC revealed a 175 bp deletion followed by many other five to ten bp deletions and a single insertion of a T nucleotide in TN 90LC relative to K326.

PCR primers EGY1-F and EGY1-R designed to amplify only a segment of NtEGY1 and to detect the 8 bp deletion in TN 90LC were tested on K326, TN 90LC, and the parents of the two mapping populations (BWDH8, BWDH16, NC 1426-17). The 8 bp deletion was found to be present in TN 90LC and also BWDH8, but not in BWDH16. Genotyping of 1056 BC₁F₁ plants from BWDH8/NC1426-17//NC1426-17 BC₁F₁ population (which segregates 1:1 for the normal:chlorophyll-deficient phenotype) for this deletion revealed complete cosegregation between the chlorophyll deficiency and the 8 bp deletion, thus highly suggesting NtEGY1 to be the gene at one of the two Yb loci.

To determine the predicted amino acid sequence differences between the wild-type and mutant NtYb₂ (NtEGY1) alleles, the corresponding cDNA strands were isolated and sequenced from K326 and TN 90LC. Because of the high degree of sequence similarity between NtEGY1 and NtEGY2, it was assumed that cDNAs of both were being simultaneously amplified. PCR products were separated using gel electrophoresis and fragments were cloned and sequenced. It was possible to distinguish cDNAs corresponding to NtEGY1 from NtEGY2 based upon the presence of a 9 bp insertion in the former that was also predicted based upon the genomic sequence in GenBank.

Analysis of the NtYb₂ cDNA sequences revealed a T→C substitution at base 618, a A→T substitution at base 621, and an 8 bp deletion of bases 623 to 630 (exon 2) in TN90, which causes a frame-shift and the creation of a premature stop codon, resulting in a truncated protein that is missing a C-terminal 330 amino acid region (FIG. 2).

In comparing the NtEGY2 cDNA sequences from TN 90LC and K326, the only difference that was found was an insertion of a T nucleotide at base 1448 of the cDNA (exon 9) in TN 90LC. This insertion causes a frame-shift and creates a premature stop codon, resulting in a truncated protein that is missing a C-terminal 42 aa region. The wild-type NtEGY1 and NtEGY2 cDNA are predicted to produce translated proteins with 97% amino acid similarity (FIG. 2).

Because of the identification of a 1 bp insertion leading to a predicted truncated NtEGY2 protein, it was hypothesized that NtEGY2 might correspond to NtYb₁. KASP primers, EGY2_kasp, were designed to detect the T insertion. Because of the extremely high degree of sequence similarity between NtEGY1 and NtEGY2 in the vicinity of the 1 bp insertion, it was difficult to design primers specific to the NtEGY2 homeolog. Nested PCR was therefore carried out with primers EGY2-nF and EGY2-nR prior to genotyping of the KASP marker. After testing the marker on K326, TN 90LC, and the parents of the two mapping populations (BWDH8, BWDH16, NC 1426-17), it was determined that TN 90LC and BWDH16 carried the mutation. A total of 1040 individuals from the BWDH16/NC1426-17//NC1426-17 BC₁F₁ population were therefore genotyped with the marker and complete cosegregation was observed between the presence of the 1 bp insertion and the yellow burley phenotype. This provided extremely strong evidence that NtEGY2 also encoded for a gene at one of the Yb loci.

### Example 2—Confirmation of Genes Underlying the Yellow Burley Loci

To provide further confirmation that NtEGY1 and NtEGY2 encode for genes controlling the yellow burley phenotype, both targeted mutation and complementation approaches were carried out. For the targeted mutagenesis, Yb₁yb₁ yb₂yb₂ or yb₁yb₁ Yb₂yb₂ genotypes were first established by hybridizing BWDH8 and BWDH16 with TN 90LC. These genotypes were used so that only a single Yb gene copy would have to be interrupted to observe the predicted phenotype if the gene targeted for mutation was indeed that positioned at a Yb locus. CRISPR-cas mediated mutagenesis resulted in the regeneration of a total of 30 plants in which the normal green phenotype was converted to a chlorophyll-deficient phenotype via induced mutation. To verify targeted mutation of Yb loci in these modified plants, the targeted regions were verified for sequence disruptions. For the NtYb₁ target sites, all 15 plants that were regenerated that also exhibited the chlorophyll-deficient phenotype contained small indels or insertions resulting in frameshifts. For the NtYb₂ target sites, all 15 plants that were regenerated that also exhibited the chlorophyll-deficient phenotype also contained small indels or insertions resulting in frameshifts.

For complementation experiments, full-length NtYb₁ and full-length NtYb₂ wild-type cDNAs were individually expressed under the control of the cauliflower mosaic virus (CaMV) 35S promoter in the burley cultivar TN 90LC and the ‘yellow burley’ nearly isogenic version of flue-cured tobacco cultivar SC58 (SC58 yb₁yb₁yb₂yb₂). Twenty out of 30 independent primary 35S:NtYb₁ transformants and 20 of 30 independent primary 35S:NtYb₂ transformants exhibited complementation of the yellow burley phenotype.

### Example 3—EGY1 and EGY2 Contribute to the Level of TSNA Production in Tobacco

### a) Determining the Effect of NtYb₁ on Nitrogen Use Efficiency and Nitrogen Utilization Efficiency

Three TN 90LC 35S:NtYb₁ R₀ transformants exhibiting transgenic complementation of the chlorophyll-deficient phenotype were self-pollinated to produce three R₁ families that segregated for the presence of the incorporated transgene(s) and the chlorophyll-deficient phenotype (Table 1). White-stem and green-stem segregants from each R₁ family were compared to each other and with non-transgenic control plants for yield, N-USE, N-UTL and NO₃—N in a replicated field experiment carried out near Oxford, N.C., USA. A randomized complete block design with 20 replicates was used.

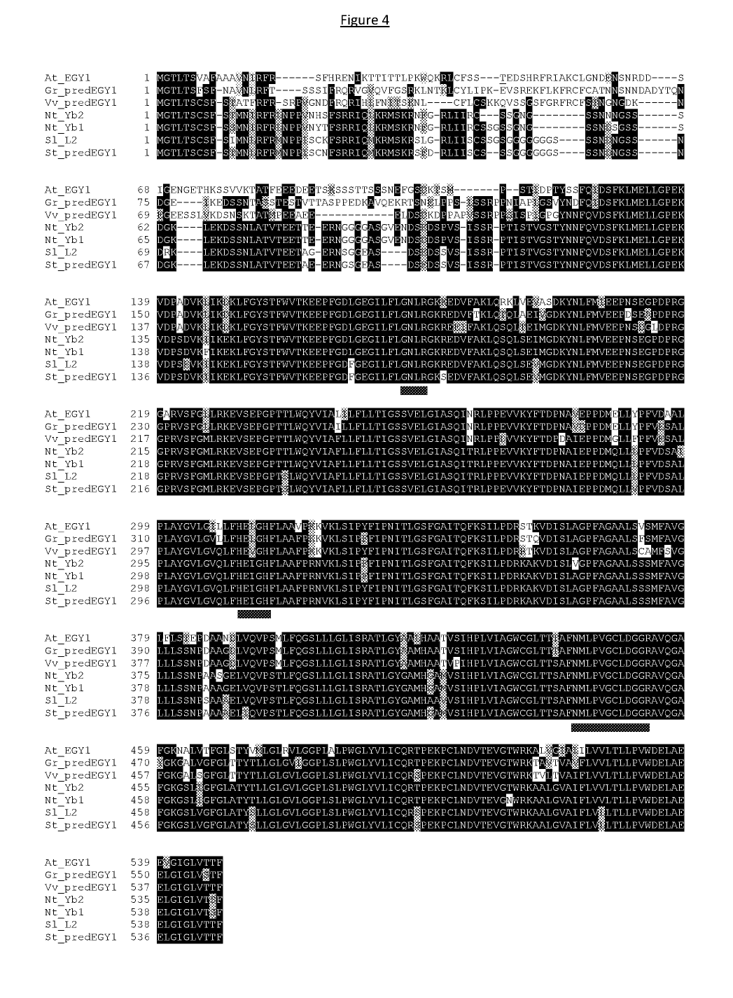

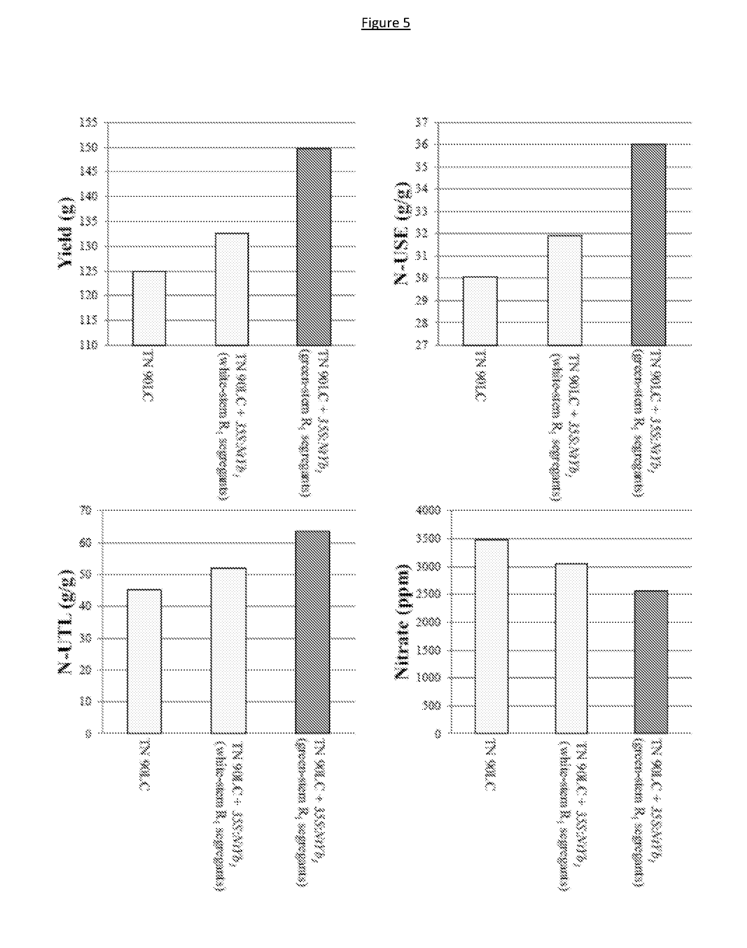

Results indicated that overexpression of NtYb₁ in TN 90LC increased plant yields, increased N-USE, increased N-UTL and decreased NO₃—N relative to the non-transgenic TN 90LC control (FIG. 5).

### b) Determining the Effect of NtYb₁ on TSNA Accumulation in Tobacco

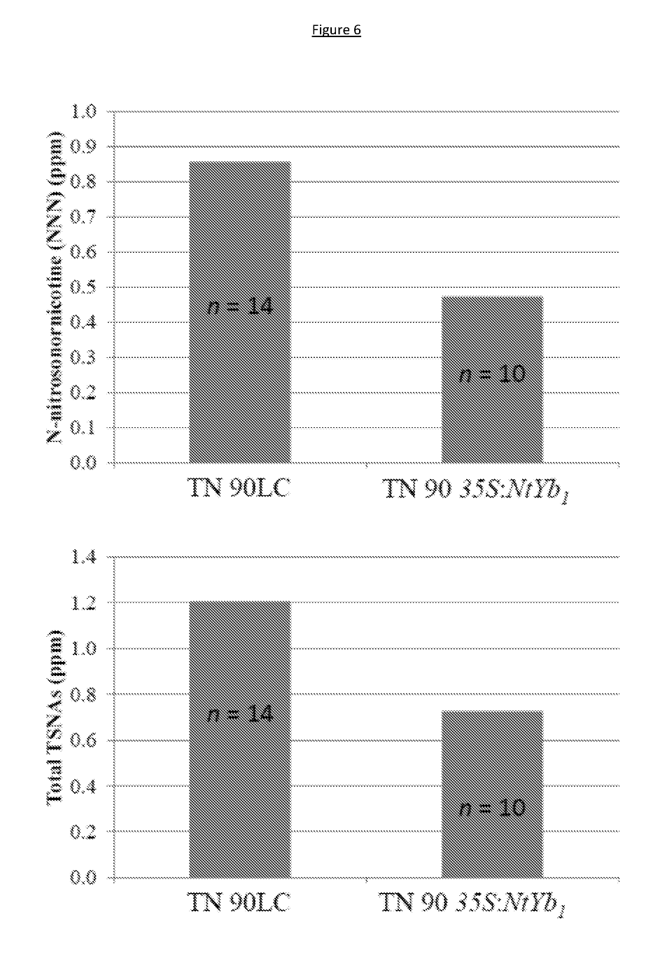

Ten green-stem TN 90LC 35S:NtYb₁ greenhouse-grown Ro transformants were compared to 14 non-transgenic control plants of greenhouse-grown TN 90LC. The transgenic 35S:NtYb₁ genotypic group was found to accumulate lower levels of N-nitrosonornicotine (NNN) and total TSNAs as compared to the non-transgenic control group (FIG. 6).

### Materials and Methods

Mapping of Yb₁ and Yb₂

The recessive yb₁ and yb₂ alleles were transferred from burley tobacco cultivar Ky 16 to flue-cured tobacco (Yb₁ Yb₁ Yb₂ Yb₂) cultivars SC58, NC95, and Coker 139 using eight cycles of backcrossing followed by multiple generations of self-pollination to establish homozygosity for the yb₁ and yb₂ alleles conferring the chlorophyll deficient phenotype. DNA was isolated from the six genotypes using a modified cetyltrimethylammonium bromide procedure (Afandor et al., 1993, Annu Rep Bean Improv Coop 36: 10-11) and genotyped with a 30 K Infinium iSelect HD BeadChip SNP chip. Genomic regions containing polymorphisms that differentiated the nearly isogenic lines were identified and corresponding SNP markers of interest were converted to Kompetitive Alle Specific PCR (KASP) markers (Semagn et al., 2014, Mol Breeding 33: 1-14).

To develop mapping populations for fine mapping both yb₁ and yb₂, a set of doubled haploid (DH) lines segregating for the yellow burley phenotype was generated by hybridizing burley tobacco cultivar Ky 14 with flue-cured tobacco cultivar K 346, isolating haploid plants via pollination of the F₁ hybrid with N. africana, and doubling the chromosome number of resulting haploid plants. Doubled haploid lines BWDH6 and BWDH8 were subsequently found to be homozygous for a dominant Yb allele at only one locus, while line BWDH16 was found to be homozygous for the dominant allele at the opposite Yb locus.

To ultimately develop progenies suitable for fine mapping Yb₁ and Yb₂, BWDH8 and BWDH16 (of either the Yb₁Yb₁ yb₂yb₂ or yb₁yb₁ Yb₂Yb₂ genotype) were both hybridized with burley tobacco breeding line NC1426-17 (yb₁yb₁ yb₂yb₂). Corresponding F₁ hybrids were then backcrossed to NC1426-17 to develop progenies that were expected to segregate 1:1 for the yellow burley phenotype. Approximately 1000 BC₁F₁ progeny from each family were grown in a field, scored for the chlorophyll-deficient phenotype, and genotyped with KASP markers corresponding to SNPs found to be closely linked to either Yb₁ or Yb₂.

SNP markers found to be closely linked to either Yb₁ or Yb₂ were aligned to a N. tabacum draft sequence to identify scaffolds that might contain Yb₁ or Yb₂ candidate genes. Genes predicted to be involved in nitrogen assimilation, nitrogen use physiology, or chlorophyll maintenance were considered as potential candidate genes underlying the yellow burley phenotype. GenBank sequences for flue cured cultivar K326 (Yb₁ Yb₁ Yb₂ Yb₂) and burley tobacco cultivar (yb₁yb₁ yb₂yb₂) were investigated for polymorphisms in these candidate genes. Primers were designed to permit genotyping for polymorphisms of interest and used to genotype approximately 1200 plants from the BWDH8/NC1426-17//NC1426-17 and BWDH16/NC1426-17//NC1426-17 BC₁F₁ populations to determine the degree of linkage between the polymorphism of interest and the genes controlling the yellow burley phenotype.

Isolation and Cloning of yb₁ and yb₂ cDNA

RNA was extracted from leaf tissue of six-week old plants of K326 (Yb₁ Yb₁ Yb₂ Yb₂) and TN 90 (yb₁yb₁ yb₂yb₂) plants using the RNeasy Plant Mini Kit (Qiagen). cDNA was synthesized using the SuperScript First-Strand Synthesis System for RT-PCR with oligo(dT) (Invitrogen). The coding region of Yb candidate genes was amplified by PCR from first-strand cDNA from K 326 and TN 90 using the primers cYb-F and cYb-R that were designed based on Yb₁ and Yb₂ predicted sequences using N. tabacum draft genome sequence information. Because few nucleotide differences existed between Yb₁ and Yb₂ candidates at either the 5′ or 3′ ends, it was not possible to design primers specific to either homeolog. Bands were therefore excised from agarose gels and purified with the Monarch DNA Gel Extraction Kit (New England Biolabs). Fragments were cloned into the pCR-Blunt vector using the Zero Blunt PCR Cloning Kit (Invitrogen) and transformed into NEB 5-alpha competent E. coli cells (New England Biolabs). Sequencing of individual clones derived from each cultivar was carried out using vector primers and subsequent analysis revealed that both Yb₁ and Yb₂ candidate fragments had been amplified.

### Isolation of Yb1 and Yb2 Genomic DNA

Genomic DNA was isolated from plants of K326 and TN90 using a modified cetyltrimethylammonium bromide procedure. The genomic DNA of each candidate gene was amplified in six fragments of approximately 1250 bp each using PCR with Q5 High-Fidelity DNA polymerase (New England Biolabs). To ensure specific amplification of the desired gene, primers were designed such that the last basepair on the 3′ end was a SNP between Yb₁ and Yb₂. Fragments were excised, purified, and cloned into pCR-Blunt. Sequencing was carried out using vector primers and, for longer sequences, internal gene sequence primers.

### Sequence Analysis and Bioinformatics Resources

DNA sequences were aligned using Vector NTI-AlignX (Thermo Fisher Scienfific, Walthmam, Mass.). Individual genomic DNA fragments were concatenated into full genomic sequences using Vector NTI-ContigExpress. The prediction of genes within genomic DNA sequences was performed with the FGENESH hidden Markov model-based gene structure prediction programme (www.softberry.com). cDNA sequences were aligned to genomic sequences with Spidey (www.ncbi.nlm.nih.gov/spidey). Amino acid alignments were performed using MUSCLE (Edgar, 2004, Nucl Acids Res_32: 1792-97). Yb orthologs were identified from different organisms by BLAST searching the NCBI database or the Phytozyme database using the full-length amino acid sequences of Yb₁ or Yb₂. Multiple sequence alignment of selected results was constructed using ClustalOmega. The phylogenetic tree was reconstructed using PHYLIP (Felsenstein, 2005, PHYLIP (Phylogeny Inference Package) Version 3.6. Distributed by the author. Department of Genome Sciences, University of Washington, Seattle) and visualized with R/ape (Paradis et al., Bioinformatics (2004) 20 (2): 289-290).

Verification of Gene Function Via Overexpression of Yb Wild-Type cDNAs

Yb₁ and Yb₂ complete wild-type cDNAs with XbaI and SacI cloning linkers were synthesized based upon the K326 cDNA sequences determined above. The synthesized cDNAs of the wild-type Yb candidate genes were ligated into pB1121 for the purpose of overexpressing the gene under the control of the constitutive CaMV 35S promoter. Standard Agrobacterium-based transformation was used to introduce 35S:NtYb₁ or 35S:NtYb₂ into burley tobacco cultivar TN 90LC and the yellow burley nearly isogenic version of flue-cured tobacco cultivars SC58 (SC58 yb₁yb₁yb₂yb₂). Transformants were selected on MS media containing 100 mg L⁻¹ kanamycin and 200 mg L⁻¹ timentin and screened using PCR primers specific to the CaMV 35S promoter region. Resulting transgenic plants were observed for restoration of the normal chlorophyll-producing phenotype.

### Determining the Effect of NtYb₁ on Nitrogen Use Efficiency and Nitrogen Utilization Efficiency

Three selected TN 90LC 35S:NtYb₁ R₀ transformants exhibiting transgenic complementation of the chlorophyll-deficient phenotype were self-pollinated to produce three R₁ families that segregated for the presence of the incorporated transgene(s) and the chlorophyll-deficient phenotype. White-stem and green-stem segregants from each R₁ family were compared to each other and with non-transgenic control plants for yield per plant, NO₃—N, N-USE and N-UTL in a replicated field experiment carried out near Oxford, N.C., USA. A randomized complete block design with 20 replicates was used. Experimental units consisted of single plants representing each of the 7 experimental entries (Table 1). Row and intra-row plant spacing were 117.8 cm and 45.7 cm, respectively, and the experiment was surrounded by border plants. 700 lbs of 6-6-18 fertilizer was applied per acre within a week of transplanting. Twenty days after the first N application, 75 gallons of liquid 21-0-0-24S was applied per acre. Production practices were consistent with those used for commercial air-cured tobacco production in North Carolina.

TABLE 1
Entries in field experiment to investigate the impact of NtYb₁
overexpression on nitrogen use efficiency and nitrogen
utilization efficiency.

| Entry | Genotype |
|---|---|
| 1 | TN 90LC + 35S:NtYb₁ P3 9a; R1 Generation (white-stem |
| | segregants) |
| 2 | TN 90LC + 35S:NtYb₁ P3 9a; R1 Generation (green-stem |
| | segregants) |
| 3 | TN 90LC + 35S:NtYb₁ P9 10a; R1 Generation (white-stem |
| | segregants) |
| 4 | TN 90LC + 35S:NtYb₁ P9 10a; R1 Generation (green-stem |
| | segregants) |
| 5 | TN 90LC + 35S:NtYb₁ P2 4a; R1 Generation (white-stem |
| | segregants) |
| 6 | TN 90LC + 35S:NtYb₁ P2 4a; R1 Generation (green-stem |
| | segregants) |
| 7 | TN 90LC |

Sixty days after transplanting, individual plants were cut at the ground level and above-ground biomass was recorded. Plants were oven-dried, ground to pass through a 1-mm sieve, and analyzed for total N according to Nelson and Sommers (Agron. J. 65:109-112;1973) and NO₃—N according to CORESTA Recommended Method No. 36 (2011). N-USE and N-UTL were calculated according to Moll et al. (Agron. J. 74:562-564, 1982) and Sisson et al. (Crop Sci. 31:1615-1620, 1991) using the following formulae:

N-use efficiency (N-USE, g g⁻¹)=(plant biomass, gram plant⁻¹/units N fertilizer, g plant⁻¹), and

N-utilization efficiency (N-UTL, g g⁻¹)=(plant biomass, g plant⁻¹/N-ACC, g plant⁻¹)

where N-ACC=(plant biomass, g plant⁻¹×% N*10)/1000

Data were analyzed using standard analyses of variance and appropriate single degree of contrast statements were carried out using PROC GLM of SAS to compare entry means (SAS Institute, Cary, N.C.). A log transformation was performed on the NO₃—N data prior to analysis in order to reduce heterogeneity of error variances.

### Determining the Effect of NtYb₁ on TSNA Accumulation in Tobacco

Ten green-stem TN 90LC 35S:NtYb₁ R₀ transformants were grown in a greenhouse along with 14 non-transgenic control plants of TN 90LC. Approximately 70 days after potting, all plants were decapitated to enhance alkaloid accumulation. Ten days after decapitation, the uppermost two leaves were collected from each plant and tagged and air cured. After five weeks of air-curing, leaves were ground to pass through a 1-mm sieve and samples were quantified for nicotine, nornicotine, anatabine, and anabasine using gas chromatography, and the TSNAs, NNN, NNK, NAB, and NAT using LC-MS. Total TSNAs were calculated as the sum of NNK, NNN, NAT, and NAB. TSNA Means for the transgenic 35S::tYb₁ TN 90LC group and the non-transgenic TN 90LC group were compared using simple t-tests (Steele et al., Principles and Procedures of Statistics, A Biometrical Approach; McGraw-Hill: New York, N.Y., 1997).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1: A method for increasing nitrogen-use efficiency and/or nitrogen-utilisation efficiency in a plant comprising modifying the plant by increasing the activity or expression of an ethylene-dependent gravitropism-deficient and yellow green protein (EGY) in said plant, wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1.

2. (canceled)

3: The method according to claim 1 wherein the EGY protein is overexpressed in said plant leaves.

4: The method or use according to claim 1 comprising expressing within the plant a polynucleotide comprising a nucleic acid sequence encoding an EGY polypeptide.

5: The method according to claim 4 wherein said polynucleotide (e.g. an exogenous polynucleotide) comprises a nucleic acid sequence encoding an EGY polypeptide operably linked with a heterologous promoter for directing transcription of said nucleic acid sequence in said plant.

6: The method according to claim 5 wherein said promoter is a leaf-specific or leaf-preferred promoter.

7: The method according to claim 1 wherein the plant is a crop plant, e.g. a fruit crop, a seed crop, a legume or a nut crop.

8: The method according to claim 1 wherein the method is for reducing a level of at least one tobacco-specific nitrosamine (TSNA) or a precursor thereto (e.g. leaf nitrate) in a tobacco plant.

9: A method for producing a plant, tobacco plant, plant propagation material, a tobacco plant propagation material, plant part, tobacco plant part, a tobacco leaf, a cut harvested tobacco leaf, a processed tobacco leaf, a cut and processed tobacco leaf, a plant product, or tobacco plant product, which has a reduction in at least one TSNA or a precursor thereto, the method comprising modifying said tobacco plant to increase expression of an ethylene-dependent gravitropism-deficient and yellow green protein (EGY) gene or increase activity of an EGY protein in said plant, wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1.

10: The method according to claim 44, wherein at least one TSNA or a precursor thereto is reduced when compared to a tobacco plant which has not been modified to increase expression of an EGY gene or increase activity of an EGY protein in said plant.

11: The method according to claim 44, wherein the TSNA is nicotine-derived nitrosamine ketone (NNK), nitrosonornicotine (NNN), nitrosoanatabine (NAT) or N-nitrosoanabasine (NAB).

12: The method according to claim 44, wherein the plant is from the species Nicotiana tabacum or Nicotiana rustica.

13: The method according to claim 1, wherein the polynucleotide encoding said EGY polypeptide:
a. comprises a polynucleotide sequence encoding the polypeptide of SEQ ID NO: 2 or 3 or a functional fragment thereof or a sequence which has at least 70% sequence identity to SEQ ID NO: 2 or 3 or a functional fragment thereof; or
b. comprises & the polynucleotide sequence shown as SEQ ID NO: 4, 5, 21 or 22 or a polynucleotide sequence which has at least 70% sequence identity to SEQ ID NO: 4, 5, 21 or 22; or
c. comprises a polynucleotide sequence which can hybridize to the polynucleotide as defined in a., or b. above.

14: The method of claim 1, wherein the polypeptide encoded by the EGY gene or the nucleic acid sequence encoding said EGY polypeptide comprises the sequence shown as SEQ ID NO: 2 or 3 or a functional fragment thereof or a sequence which has at least 70% (preferably 85%, more preferably 90%) sequence identity to SEQ ID NO: 2 or 3 or a functional fragment thereof.

15: The construct or vector comprising a nucleic acid encoding an EGY protein as defined in claim 13.

16: The construct or vector comprising a nucleic acid encoding an EGY protein as defined in claim 13 operably linked with a leaf-specific or leaf-preferred promoter.

17: A plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product:
i) comprising an exogenous EGY polynucleotide wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1;
ii) comprising a construct or vector according to claim 15; and/or
iii) obtainable by a method according to claim 1.

18-19. (canceled)

20: The plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product of claim 17, comprising harvested leaf of a plant.

21: The harvested leaf of claim 20, wherein the harvested leaf is a cut harvested leaf.

22: A processed leaf:
a. comprising a plant cell according to claim 17;
b. obtainable from a plant obtainable from a method comprising modifying the plant by increasing the activity or expression of an ethylene-dependent gravitropism-deficient and yellow green protein (EGY) in said plant, wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1;
c. obtainable from processing a plant according to claim 17;
d. obtainable from a plant propagated from a plant propagation material according to claim 17; and/or
e. obtainable by processing a harvested leaf according to claim 17.

23: The processed leaf according to claim 22, wherein the plant or leaf is processed by curing, fermentation, pasteurising or combinations thereof.

24: The processed leaf according to claim 22 wherein the processed leaf is cut processed leaf.

25: The plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product according to claim 17, wherein said plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product comprises a crop plant.

26: The plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product according to claim 17, wherein said plant, tobacco plant, plant cell, tobacco plant cell, plant propagation material, a tobacco plant propagation material, plant part, tobacco plant part, a tobacco leaf, a cut harvested tobacco leaf, a processed tobacco leaf, a cut and processed tobacco leaf a plant product, or tobacco plant product is from the species Nicotiana tabacum or Nicotiana rustica.

27: A plant product:
a. prepared from a plant obtained or obtainable by a method comprising modifying the plant by increasing the activity or expression of an ethylene-dependent gravitropism-deficient and yellow green protein (EGY) in said plant, wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1
b. prepared from a plant according to claim 17;
c. prepared from a plant propagated from the plant propagation material according to claim 17;
d. prepared from a harvested leaf according to claim 17;
e. prepared from a processed leaf according to claim 17; and/or
f. prepared from or comprising a plant extract obtained or obtainable from a modified plant according to claim 17.

28: The plant product according to claim 27 which is a consumable plant product.

29: The consumable plant product according to claim 28 which is a tobacco product.

30: The tobacco product according to claim 29 wherein the tobacco product is selected from a smoking article, a smokeless tobacco product or a tobacco heating device comprising said plant product.

31-32. (canceled)

33: A plant extract of a plant or plant part according to 17.

34-35. (canceled)

36: The method of claim 9, further comprising breeding said plant.

37: The method of claim 9, further comprising growing a crop of said plant.

38. (canceled)

39: A method for screening a plant for an EGY variant or identifying a plant with a predisposition to increased nitrogen use efficiency (NUE) and/or increased nitrogen utilization efficiency (NUTL), the method comprising, comparing an EGY polynucleotide sequence (e.g. gene) in said plant to a wild-type EGY polynucleotide sequence to identify the presence of a variant EGY polynucleotide sequence in said plant, and optionally further determining that said variant EGY polynucleotide sequence increases NUE and/or NUTL compared to a plant which comprises the wild-type EGY polynucleotide sequence, wherein said EGY protein has the following conserved residues: a GNLR motif comprising Gly-176, Asn-177, Leu-178 and Arg-179; a HEXXH motif comprising His-311, Glu-312, X-313, X-314 and His-315; and a NXXPXXXLDG motif comprising Asn-442, X-443, X-444, Pro-445, X-446, X-447, X-448, Leu-449, Asp-450 and Gly-451; wherein X is any amino acid and wherein the numbering of the conserved residues means the effective equivalent position of the EGY protein is aligned with the Arabidopsis thaliana EGY1 protein having SEQ ID NO: 1.

40. (canceled)

41: The method according to claim 39, wherein said plant is a tobacco plant and said wild-type EGY polynucleotide sequence is the sequence shown as SEQ ID NO: 4, 5, 21 or 22.

42: The method according to claim 41, further comprising determining that said variant EGY polynucleotide sequence is associated with reduced TSNA levels in said plant compared to a plant which comprises the wild-type EGY polynucleotide sequence.

43: A method of producing a plant having increased nitrogen use efficiency (NUE) and/or increased nitrogen utilization efficiency (NUTL), comprising:
a. crossing a donor plant identified as having increased NUE and/or increased NUTL by a method according to claim 39 with a recipient plant that does not have said variant EGY polynucleotide sequence and possesses commercially desirable traits;
b. optionally isolating genetic material from a progeny of said donor plant crossed with said recipient plant; and
c. optionally performing molecular marker-assisted selection with a molecular marker comprising identifying an introgressed region comprising the variant EGY polynucleotide sequence which is associated with increased NUE and/or increased NUTL.

44: The method of claim 9, comprising a tobacco plant, tobacco plant propagation material, tobacco plant part, a tobacco leaf, a cut harvested tobacco leaf, a processed tobacco leaf or a cut and processed tobacco leaf, or tobacco plant product.
